(19) United States
(12) Patent Application Publication
(10) Pub. No.: US 2016/0114338 A1
(43) Pub. Date: Apr. 28, 2016

(54) CATIONIC COLLECTORS WITH MIXED POLYAMIDOAMINES AND METHODS FOR MAKING AND USING SAME

(71) Applicant: Georgia-Pacific Chemicals LLC, Atlanta, GA (US)

(72) Inventor: David R. Snead, Atlanta, GA (US)

(73) Assignee: GEORGIA-PACIFIC CHEMICALS LLC, Atlanta, GA (US)

(21) Appl. No.: 14/920,844

(22) Filed: Oct. 22, 2015

Related U.S. Application Data
(60) Provisional application No. 62/067,672, filed on Oct. 23, 2014.

(51) Int. Cl. B03D 3/06; C07C 233/36; C07C 233/37

(52) U.S. Cl. CPC B03D 3/06; C07C 233/37; C07C 233/36; C07C 2101/08

(57) ABSTRACT

Compositions that include a polyamidoamine, aqueous mixtures that include the polamidoamine and an ore, and methods for making and using same. The composition can include a polyamidoamine having the chemical formula (A). In the chemical formula (A), R¹ and R² can be different and can be selected from a saturated or unsaturated, substituted or unsubstituted, linear or branched, cyclic, heterocyclic, or aromatic hydrocarbyl group, R³ and R⁴ can independently be hydrogen or a saturated or unsaturated, substituted or unsubstituted, linear or branched, cyclic, heterocyclic, or aromatic hydrocarbyl group, each m can be an integer of 1 to 5, and n can be an integer of 2 to 8. The aqueous mixture can include an ore, water, and the composition.

### CROSS-REFERENCE TO RELATED APPLICATION

This application claims priority to U.S. Provisional Patent Application No. 62/067,672, filed on Oct. 23, 2014, which is incorporated by reference herein.

### BACKGROUND

1. Field

Embodiments described generally relate to compositions that can include a polyamidoamine and methods for making and using same. More particularly, such embodiments relate to compositions that include a polyamidoamine, aqueous mixtures that include the polyamidoamine and an ore, and methods for making and using same.

2. Description of the Related Art

Froth flotation is a method that uses the differences in the hydrophobicity of the mineral particles to be separated or purified from aqueous slurries containing the mineral particles and one or more impurities. Certain heteropolar or nonpolar chemicals called collectors are typically added to the aqueous slurries to enhance or form water repellencies on the surfaces of these mineral particles. These collectors are designed to selectively attach to one or more of the mineral particles to be separated and form a hydrophobic monolayer on the surfaces of the mineral particles. The formation of the hydrophobic monolayer lowers the surface energy of the mineral particles, which increases the chance that the particles will bind with air bubbles passing through in the slurry. The density of the combined air bubble and mineral particles is less than the displaced mass of the aqueous slurry, which causes the air bubble and mineral particles to float to the surface of the slurry. A mineral-rich froth is formed by the collection of the floating air bubble and mineral particles at the surface of the slurry that can be skimmed off from the surface, while other minerals or material, e.g., impurities, remain submerged and/or flocculated in the slurry. The flotation of minerals with a negative surface charge, such as silica, silicates, feldspar, mica, clays, chrysocolla, potash and others, from an aqueous slurry can be achieved using cationic collectors.

In reverse flotation, impurities are floated out of and away from the unpurified or crude materials to be beneficiated or otherwise purified. In particular, phosphate minerals, iron ore, copper ores, and other minerals and/or ores are frequently beneficiated in this manner. In many cases, silicate is the main component of the mineral impurities that cause quality reductions in the purified product. The minerals containing silicates or other silicon oxides include quartz, sand, mica, feldspar, muscovite, and biotite. A high silicate content lowers the quality of the phosphate or other purified material.

Phosphorous ores generally contain impurities and phosphate materials, e.g., calcium phosphate that can be represented by the general chemical formula Ca₅(PO₄)₃(X), where X can be fluoride, chloride, and/or hydroxide. Phosphate materials, such as calcium phosphate, generally have a polar, hydrophilic surface. Many of the impurities, e.g., silicates, in the phosphorus ore also have polar, hydrophilic surfaces and are not easy to selectively separate from the phosphate material. Conventional collectors used for silicate flotation in phosphate beneficiation generally exhibit inadequate results with respect to selectivity and yield of phosphate relative to the impurities.

Monoamidoamines have been used in phosphate beneficiation, but are difficult to handle and use as a collector due to generally being highly viscous liquids or waxy solids at room temperature, e.g., about 25° C. Monoamidoamines also exhibit inadequate selectivity of silicate over phosphate and, therefore, provide a phosphate product with a higher impurity content than other conventional collectors. In addition to lower purity, phosphate products recovered with monoamidoamines generally are recovered in lower yields relative to other conventional collectors.

There is a need, therefore, for improved collectors and methods for making and using same.

### SUMMARY

Compositions that include a polyamidoamine, aqueous mixtures that include the polamidoamine and an ore, and methods for making and using same are provided. In one or more embodiments, the composition can include a polyamidoamine having the chemical formula:

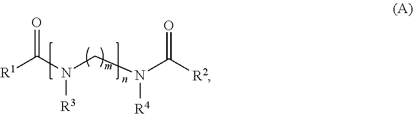

where R¹ and R² can be different and can be selected from a saturated or unsaturated, substituted or unsubstituted, linear or branched, cyclic, heterocyclic, or aromatic hydrocarbyl group, R³ and R⁴ can independently be hydrogen or a saturated or unsaturated, substituted or unsubstituted, linear or branched, cyclic, heterocyclic, or aromatic hydrocarbyl group, each m can be an integer of 1 to 5, and n can be an integer of 2 to 8.

In one or more embodiments, the aqueous mixture can include an ore; water; and a polyamidoamine having the chemical formula (A), where R¹ and R² can be different and can be selected from a saturated or unsaturated, substituted or unsubstituted, linear or branched, cyclic, heterocyclic, or aromatic hydrocarbyl group, R³ and R⁴ can independently be hydrogen or a saturated or unsaturated, substituted or unsubstituted, linear or branched, cyclic, heterocyclic, or aromatic hydrocarbyl group, each m can be an integer of 1 to 5, and n can be an integer of 2 to 8.

In one or more embodiments, a method for purifying an ore can include combining an ore, water, and a polyamidoamine to produce an aqueous mixture. The ore can include an impurity. The polyamidoamine can have the chemical formula (A), where R¹ and R² can be different and can be selected from a saturated or unsaturated, substituted or unsubstituted, linear or branched, cyclic, heterocyclic, or aromatic hydrocarbyl group, R³ and R⁴ can independently be hydrogen or a saturated or unsaturated, substituted or unsubstituted, linear or branched, cyclic, heterocyclic, or aromatic hydrocarbyl group, each m can be an integer of 1 to 5, and n can be an integer of 2 to 8. A flocculated material that can include the impurity and the polyamidoamine from the aqueous mixture can be collected. A purified ore that contains less of the impurity than the ore can also be collected from the aqueous mixture.

### DETAILED DESCRIPTION

It has been surprisingly and unexpectedly discovered that compositions containing one or more polyamidoamines that have two or more amido groups with different hydrocarbyl groups provide high yields and/or selectivity by impurity, e.g., silicate, flotation in an aqueous mixture for the purification or beneficiation of one or more ores. For example, the compositions containing the polyamidoamines surprisingly and unexpectedly perform better, e.g., greater yield and/or selectivity, in phosphate beneficiation than monoamidoamines. Without wishing to be bound by theory, it is believed that the compositions containing one or more polyamidoamines that have two or more amido groups with different hydrocarbyl groups provide enhanced adhesion to the surfaces of impurities, e.g., silicate particles and other gangue material, which lowers the surface energy of the impurities. This reduced surface energy increases the likelihood for the impurities to bind or otherwise attract to air bubbles and thus increases the buoyancy of the impurities. The purified ores can be collected or removed from the aqueous mixture, for example, after settling toward or on a bottom of a separation vessel. Accordingly, the compositions can be used as cationic collectors.

The polyamidoamine can be or include one or more amidoamines having the chemical formula (A):

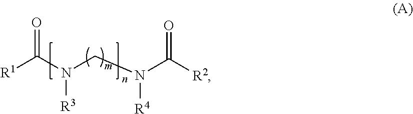

where R¹ and R² can be different and can be selected from a saturated or unsaturated, substituted or unsubstituted, linear or branched, cyclic, heterocyclic, or aromatic hydrocarbyl group, R³ and R⁴ can independently be hydrogen or a saturated or unsaturated, substituted or unsubstituted, linear or branched, cyclic, heterocyclic, or aromatic hydrocarbyl group, each m can be an integer of 1, 2, 3, 4, 5, 6, 7, 8, or greater, and n can be an integer of 1, 2, 3, 4, 5, 6, 7, 8, 9, 10, 11, 12, or greater.

In some examples, R¹, R², R³, and R⁴ can independently be an alkyl, an alkenyl, an alkynyl, an aryl, an alkoxyl, a carboxylic acid, an amino, an amido, a saturated and/or unsaturated fatty acid group, and/or isomers thereof. R¹, R², R³, and R⁴ can independently be a hydrocarbyl group with 1, 2, 3, 4, 5, 6, 7, 8, 9, 10, 11, 12, 13, 14, 15, 16, 17, 18, 19, 20, 21, 22, 25, 30, or more carbon atoms. For example, R¹, R², R³, and R⁴ can independently be a C4 to C30 chain, a C8 to C24 chain, a C9 to C30 chain, a C9 to C24 chain, a C9 to C21 chain, a C9 to C20 chain, a C9 to C19 chain, a C9 to C18 chain, a C9 to C17 chain, a C9 to C15 chain, a C10 to C24 chain, a C10 to C20 chain, a C10 to C18 chain, a C11 to C21 chain, a C11 to C19 chain, a C11 to C17 chain, a C12 to C20 chain, a C14 to C20 chain, a C14 to C19 chain, a C14 to C18 chain, a C14 to C17 chain, a C14 to C16 chain, a C14 to C15 chain, a C15 to C20 chain, a C15 to C19 chain, a C15 to C18 chain, a C15 to C17 chain, or a C15 to C16 chain.

In one or more examples, R¹, R², R³, and R⁴ can independently be derived from one or more fatty acid sources. Illustrative fatty acid sources can be or include, but are not limited to, one or more fatty acids, tall oil fatty acids (TOFA), rosin acids, crude tall oils (CTO), distilled tall oils (DTO), tall oil pitches, portions thereof, fractions thereof, or any mixture thereof. Other illustrative fatty acid sources can be or include lauric acid, stearic acid, isostearic acid, naphthenic acid, oleic acid, linoleic acid, linolenic acid, palmitic acid, salts thereof, isomers thereof, or any mixture thereof. In some examples, R³ and R⁴ can both be hydrogen and R¹ and R² can independently be derived from lauric acid, stearic acid, isostearic acid, naphthenic acid, oleic acid, linoleic acid, linolenic acid, palmitic acid, other fatty acids, isomers thereof, or any mixture thereof.

In some examples of polyamidoamines, R¹, R², R³, and R⁴ can independently have all saturated bonds, such as saturated fatty acid groups, and therefore no unsaturated bonds. In other examples of polyamidoamines, R¹, R², R³, and R⁴ each can independently have one or more unsaturated bonds, such as unsaturated fatty acid groups. For example, R¹, R², R³, and R⁴ can independently have 0, 1, 2, 3, 4, 5, 6, 7, 8, 9, 10, 11, 12, 15, 20, or more unsaturated bonds. In some examples, R¹, R², R³, and R⁴ can independently have less than 10 unsaturated bonds, less than 8 unsaturated bonds, less than 6 unsaturated bonds, or less than 5 unsaturated bonds, such as, for example, 0, 1, 2, 3, or 4 unsaturated bonds.

In some examples, R¹ and R² can be different and can be a C6 to C24 chain having 0, 1, 2, 3, 4, 5, or more unsaturated bonds. For example, R¹ and R² can be a C8 to C24 chain having 0 to 5, 0 to 4, 0 to 3, or 0 to 2 unsaturated bonds. In other examples, R¹ and R² can be a C10 to C18 chain having 0 to 3 unsaturated bonds. In other examples, R¹ and R² can be C₉H₁₉, C₉H₁₇, C₉H₁₅, C₉H₁₃, C₁₁H₂₃, C₁₁H₂₁, C₁₅H₃₃, C₁₅H₃₁, C₁₅H₂₉, C₁₇H₃₅, C₁₇H₃₃, C₁₇H₃₁, C₁₇H₂₉, C₁₉H₃₇, C₁₉H₃₅, C₁₉H₃₃, C₁₉H₃₁, C₁₉H₂₉, isomers thereof, combinations thereof, or any mixture thereof In some examples, R¹ and R² can be the —CH₂CH₂(C₅H₈)CH₂CH₃ hydrocarbyl, such as derived from naphthenic acid. R³ and R⁴ can independently be hydrogen or a C6 to C24 chain having 0, 1, 2, 3, 4, 5, or more unsaturated bonds. For example, R³ and R⁴ can independently be hydrogen. In other examples, R³ and R⁴ can independently be a C8 to C24 chain having 0 to 3 unsaturated bonds. In other examples, R³ and R⁴ can independently be hydrogen, an amino, an amido, or a C10 to C18 chain having 0 to 3 unsaturated bonds.

The value of m defines number of carbon atoms, i.e., the carbon chain length, of the organic diyl group having the N(R³)(CH₂)ₘ portion of the chemical formula (A). In one or more examples, each m can be 1, 2, 3, 4, 5, 6, 7, 8, or greater. In some examples, each m can be 1 to 8, 1 to 7, 1 to 6, 1 to 5, 1 to 4, 1 to 3, or 1 to 2. For example, each m can be 1, 2, 3, 4, 5, 6, 7, or 8, and the organic diyl group having the N(R³)(CH₂)ₘ portion of the chemical formula (A) can be or include methanediyl (—CH₂—), ethanediyl (—CH₂CH₂—), propanediyl (—CH₂CH₂CH₂—), butanediyl (—CH₂(CH₂)₂CH₂—), pentanediyl (—CH₂(CH₂)₃CH₂—), hexanediyl (—CH₂(CH₂)₄CH₂—), heptanediyl (—CH₂(CH₂)₅CH₂—), octanediyl (—CH₂(CH₂)₆CH₂—), or isomers thereof, respectively. In some specific examples, each m can be 1, 2, 3, or 4, and the organic diyl group having the N(R³)(CH₂)ₘ portion of the chemical formula (A) can include methylene, ethylene, propylene, or butylene, respectively.

The value of n defines number of organic diyl groups having the N(R³)(CH₂)ₘ portion of the chemical formula (A). In one or more examples, n can be 1, 2, 3, 4, 5, 6, 7, 8, 9, 10, 11, 12, or greater. In some examples, n can be 1 to 12, 1 to 10, 1 to 8, 1 to 6, 1 to 5, 1 to 4, 1 to 3, 1 to 2, 2 to 12, 2 to 10, 2 to 8, 2 to 6, 2 to 5, 2 to 4, or 2 to 3. For example, n can be 2, 3, 4, or 5 and the polyamidoamines can be or include diamidomonoamines, diamidodiamines, diamidotriamines, or diamidotetraamines, respectively. In other examples, the polyamidoamines can be or include triamidomonoamines, triamidodiamines, triamidotriamines, or triamidotetraamines.

In one example, R³ in each of the organic diyl groups having the N(R³)(CH₂)ₘ portion of the chemical formula (A) contained in a single polyamidoamine molecule can be the same group or can independently be different groups with respected to one another. Therefore, each of the R³ groups can independently be hydrogen or a saturated or unsaturated, substituted or unsubstituted, linear or branched, cyclic, heterocyclic, or aromatic hydrocarbyl group. Similarly, each m can independently be selected for each organic diyl group. For example, if n is 2, then the organic diyl groups having the N(R³)(CH₂)ₘ portion is N(R³)(CH₂)ₘN(R³)(CH₂)ₘ, and the polyamidoamine can include N(H)(CH₂)ₘN(H)(CH₂)ₘ (if both R³'s are hydrogen), N(CH₃)(CH₂)ₘN(CH₃)(CH₂)ₘ (if both R³'s are methyl), N(H)(CH₂)ₘN(CH₃)(CH₂)ₘ (if one R³ is hydrogen and one R³ is methyl), or any other permutation.

In another example, R¹, R², R³, and R⁴ can independently be or include one or more amino groups, one or more amido groups, or one or more amidoamino groups. For example, R¹, R², R³, and R⁴ can independently be or include one or more amido groups and the polyamidoamines can include triamidoamines, tetraamidoamines, pentamidoamines, or higher polyamidoamines.

In some examples, the polyamidoamines can be or include one or more amidoamines, where R¹ and R² can be different and can be selected from a C8 to C24 chain having 0 to 3 unsaturated bonds, R³ and R⁴ can be hydrogen, each m can be an integer of 2 to 4, and n can be an integer of 2 to 5. For example, R¹ and R² can be different and can be selected from C₉H₁₉, C₉H₁₇, C₉H₁₅, C₉H₁₃, C₁₁H₂₃, C₁₁H₂₁, C₁₅H₃₃, C₁₅H₃₁, C₁₅H₂₉, C₁₇H₃₅, C₁₇H₃₃, C₁₇H₃₁, C₁₇H₂₉, C₁₉H₃₇, C₁₉H₃₅, C₁₉H₃₃, C₁₉H₃₁, or C₁₉H₂₉. In other examples, the polyamidoamines can be or include one or more amidoamines where R¹ and R² can be different and can be selected from a C10 to C18 chain having 0 to 3 unsaturated bonds, R³ and R⁴ can be hydrogen, each m can be an integer of 2 or 3, and n can be an integer of 2, 3, or 4. For example, R¹ and R² can be C₁₁H₂₃, C₁₁H₂₁, C₁₅H₃₃, C₁₅H₃₁, C₁₅H₂₉, C₁₇H₃₅, C₁₇H₃₃, C₁₇H₃₁, or C₁₇H₂₉, and n can be 2.

In some illustrative polyamidoamines, m can be 2, where the organic diyl group having the N(R³)(CH₂)ₘ portion of the chemical formula (A) can include an ethanediyl or ethylene group. These polyamidoamines can be referred to as polyethylenepolyamidoamines and can have the chemical formula (B):

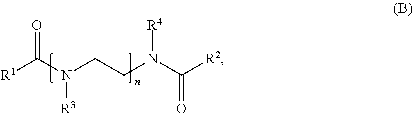

where R¹, R², R³, R⁴, and n are defined as above for the chemical formula (A).

Polyethylene polyamidoamines can include polyethylene diamidoamines, polyethylene triamidoamines, and polyethylene polyamidoamines with four or more amido groups. In some examples of polyamidoamines having the chemical formula (B), R¹ and R² can be different and can be selected from a C8 to C24 chain having 0 to 5 or 0 to 3 unsaturated bonds or a C10 to C18 chain having 0 to 5 or 0 to 3 unsaturated bonds. In other examples of polyamidoamines having the chemical formula (B), R¹ and R² can be different and can be selected from C₉H₁₉, C₉H₁₇, C₉H₁₅, C₉H₁₃, C₁₁H₂₃, C₁₁H₂₁, C₁₅H₃₃, C₁₅H₃₁, C₁₅H₂₉, C₁₇H₃₅, C₁₇H₃₃, C₁₇H₃₁, C₁₇H₂₉, C₁₉H₃₇, C₁₉H₃₅, C₁₉H₃₃, C₁₉H₃₁, or C₁₉H₂₉. In some examples of polyamidoamines having the chemical formula (B), R³ and R⁴ can independently be hydrogen, an amino, an amido, or a C10 to C18 chain having 0 to 3 unsaturated bonds. In some examples of polyamidoamines having the chemical formula (B), n can be 1, 2, 3, 4, 5, 6, 7, or 8. For example, n can be 2 to 8, 2 to 5, 2 to 4, or 2 to 3.

In some specific examples, the polyamidoamines can have the chemical formula (B), where R¹ and R² can be different and can be selected from a C8 to C24 chain, R³ and R⁴ can be hydrogen, and n can be 2, 3, 4, or 5. In other specific examples, the polyamidoamines can have the chemical formula (B), where R¹ and R² can be different and can be selected from a C10 to C18 chain, R³ and R⁴ can be hydrogen, and n can be 2, 3, or 4.

In other illustrative polyamidoamines, R³ and R⁴ can be hydrogen in the chemical formula (A). The polyamidoamines, therefore, can be or include one or more amidoamines having the chemical formula (C):

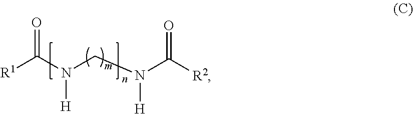

where R¹, R², m, and n are defined as above for the chemical formula (A).

In some specific examples, the polyamidoamines can have the chemical formula (C), where R¹ and R² can be different and can be selected from a C8 to C24 chain, each m can be 2, 3, or 4, and n can be 2, 3, 4, or 5. In other specific examples, the polyamidoamines can have the chemical formula (C), where R¹ and R² can be a C10 to C18 chain, each m can be 2 or 3, and n can be 2, 3, or 4.

In other illustrative polyamidoamines, R³ and R⁴ can be hydrogen and m can be 2, where the organic diyl group having the N(R³)(CH₂)ₘ portion of the chemical formula (A) can include an ethanediyl or ethylene group. These polyethylenepolyamidoamines can have the chemical formula (D):

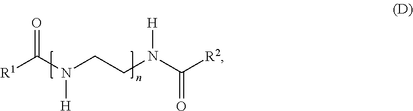

where R¹, R², and n are defined as above for the chemical formula (A).

In some specific examples, the polyamidoamines can have the chemical formula (D), where R¹ and R² can be different and can be selected from a C8 to C24 chain and n can be 2, 3, 4, or 5. In other specific examples, the polyamidoamines can have the chemical formula (D), where R¹ and R² can be C₉H₁₉, C₉H₁₇, C₉H₁₅, C₉H₁₃, C₁₁H₂₃, C₁₁H₂₁, C₁₅H₃₃, C₁₅H₃₁, C₁₅H₂₉, C₁₇H₃₅, C₁₇H₃₃, C₁₇H₃₁, C₁₇H₂₉, C₁₉H₃₇, C₁₉H₃₅, C₁₉H₃₃, C₁₉H₃₁, or C₁₉H₂₉ and n can be 2, 3, or 4. In other specific examples, the polyamidoamines can have the chemical formula (D), where R¹ and R² can be C₉H₁₅, C₉H₁₃, C₁₁H₂₃, C₁₁H₂₁, C₁₅H₃₃, C₁₅H₃₁, C₁₅H₂₉, C₁₇H₃₅, C₁₇H₃₃, C₁₇H₃₁, Or C₁₇H₂₉ and n can be 2, 3, or 4.

In other examples, the polyamidoamines can have the chemical formula (A), where R³ and R⁴ can be hydrogen, m can be 2, and n can be 2, 3, or 4, thereby providing polyethylenepolyamidoamines having the chemical formulas (E), (F), and (G), respectively:

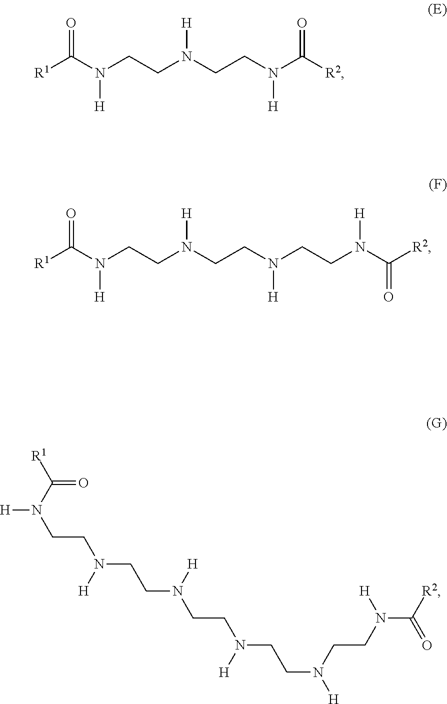

where R¹ and R² are defined as above for the chemical formula (A). In some examples of polyamidoamines having the chemical formulas (E)-(G), R¹ and R² can be different and can be selected from a C8 to C24 chain having 0 to 3 unsaturated bonds or a C10 to C18 chain having 0 to 3 unsaturated bonds. In other examples of polyamidoamines having the chemical formulas (E)-(G), R¹ and R² can be a C₉H₁₉, C₉H₁₇, C₉H₁₅, C₉H₁₃, C₁₁H₂₃, C₁₁H₂₁, C₁₅H₃₃, C₁₅H₃₁, C₁₅H₂₉, C₁₇H₃₅, C₁₇H₃₃, C₁₇H₃₁, C₁₇H₂₉, C₁₉H₃₇, C₁₉H₃₅, C₁₉H₃₃, C₁₉H₃₁, or C₁₉H₂₉. In other examples of polyamidoamines having the chemical formulas (E)-(G), R¹ and R² can be a C₉H₁₅, C₉H₁₃, C₁₁H₂₃, C₁₁H₂₁, C₁₅H₃₃, C₁₅H₃₁, C₁₅H₂₉, C₁₇H₃₅, C₁₇H₃₃, C₁₇H₃₁, or C₁₇H₂₉.

In one or more examples, m can be 2 in the chemical formula (A), and the polyamidoamines can be or include one or more polyethylenepolyamidoamines having the chemical formula (H):

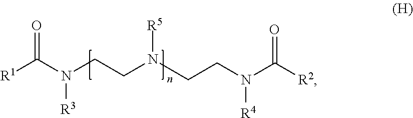

where R¹, R², R³, R⁴, and n are defined as above for the chemical formula (A), and where R⁵ can be hydrogen or a saturated or unsaturated, substituted or unsubstituted, linear or branched, cyclic, heterocyclic, or aromatic hydrocarbyl group. In some examples, R⁵ can be or include hydrogen or any hydrocarbyl group disclosed for R¹, R², R³, or R⁴. For example, R³, R⁴, and each R⁵ can independently be hydrogen or a saturated or unsaturated, substituted or unsubstituted, linear or branched, cyclic, heterocyclic, or aromatic hydrocarbyl group. In some examples, R³, R⁴, and each R⁵ can all be hydrogen. In other examples, R¹, R², R³, R⁴, and each R⁵ can independently be a hydrocarbyl group that can be or include one or more alkyl, alkenyl, alkynyl, aryl, alkoxyl, carboxylic acid, amino, amido, saturated and/or unsaturated fatty acid group, isomers thereof, combinations thereof, or mixtures thereof.

In some examples, R³, R⁴, and each R⁵ can be hydrogen in the chemical formula (H), and the polyamidoamines can be or include one or more polyethylenepolyamidoamines having the chemical formula (I):

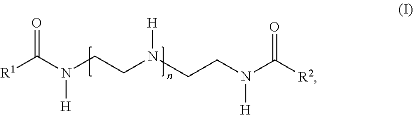

where R¹, R², and n are defined as above for the chemical formula (H).

In some examples of the polyethylenepolyamidoamines having the chemical formula (I), n can be 1, 2, 3, 4, 5, 6, 7, 8, or greater. For example, the polyamidoamines can have the chemical formula (I), where n can be 1, 2, or 3, thereby providing the above chemical formulas (E), (F), and (G), respectively.

In one or more examples, any of the polyamidoamines having the chemical formulas (A)-(I) can be combined, mixed, and/or reacted with one or more reagents to form salts, complexes, adducts, hydrates, or other forms of the polyamidoamines. For example, one or more polyamidoamines can be reacted with one or more acids to form one or more polyamidoaminates. The polyamidoamines can be reacted with the one or more reagents, such as acid, before being combined with other components to form the composition. Alternatively, the polyamidoamines and the one or more reagents can be combined as separate components, at the same time or at different times, to form the composition.

In one example, one or more organic acids can be mixed, blended, or otherwise combined with one or more polyamidoamines. Combining the organic acid with the polyamidoamine can make, form, or otherwise produce one or more salts of the polyamidoamines, e.g., polyamidoaminates. Illustrative organic acid sources or organic acids can include, but are not limited to, acetic acid, glycolic acid, lactic acid, pyruvic acid, formic acid, propionic acid, butyric acid, valeric acid (pentanoic acid), oxalic acid, malonic acid, caproic acid, enanthic acid, caprylic acid, pelargonic acid, capric acid, undecylic acid, lauric acid, isomers thereof, hydrates thereof, salts thereof, complexes thereof, adducts thereof, or any mixture thereof. In some examples, the one or more organic acids can be or include acetic acid. In other example, the one or more organic acids can be or include glacial acetic acid.

It has been surprisingly and unexpectedly discovered that combining the polyamidoamine, e.g., diamidoamines and/or triamidoamines, and the organic acid, e.g., acetic acid, produces a free flowing, homogeneous solution at room temperature, e.g., about 25° C. For example, a mixture of the polyamidoamine and organic acid, e.g., acetic acid, surprisingly and unexpectedly produces a composition that has a lower viscosity and is freer flowing at a temperature of about 25° C. than monoamidoamines or polyamidoamines free of the organic acid, which are often solids, waxy solids, or highly viscous liquids at a temperature of about 25° C.

In some examples, the polyamidoamines can be or include one or more polyamidoaminates and/or complexes of the amidoamines having the chemical formula (J):

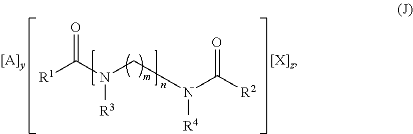

where R¹, R², R³, R⁴, each m, and n are defined as above for the chemical formula (A), and where “A” can be hydrogen, one or more alkali metals, one or more alkaline earth metals, ammonium, alkylammonium compounds, one or more hydrocarbyl groups, or one or more cationic species, “X” can be one or more conjugate bases, halides, e.g., F, Cl, Br, or I anions, hydroxide, or other anionic species, and each “y” and “z” can independently be about 0.1 to about 8, such as, for example, about 1 to about 8 or about 1 to about 4.

The A can be hydrogen, lithium, sodium, potassium, cesium, magnesium, calcium, ammonium, monoalkylammonium, dialkylammonium, trialkylammonium, tetraalkylammonium, adducts thereof, complexed salts thereof, hydrates thereof, or mixtures thereof. In some examples, the A can be bonded to the nitrogen atom of the N(R³)(CH₂)ₘ portion to produce a cationic [N(A)(R³)(CH₂)ₘ]⁺ portion of the polyamidoamine, such as a quaternary ammonium cation. The X can be one or more one or more anionic species coordinated with the cationic [N(A)(R³)(CH₂)ₘ]⁺ portion of the polyamidoamine. In some examples, the X can be one or more conjugate bases, such as organic conjugate bases, inorganic conjugate bases, or a mixture thereof. The X can be, for example, but not limited to, one or more organic conjugate bases of monocarboxylic acids, dicarboxylic acids, tricarboxylic or higher acids, amino acids, sugars, isomers thereof, hydrates thereof, salts thereof, complexes thereof, adducts thereof, or any mixture thereof. Illustrative conjugate bases can be or include, but are not limited to, acetate, glycolate, lactate, pyruvate, formate, propionate, butyrate, valerate (pentanoate), oxalate, malonate, malonate, caproate, enanthate, caprylate, pelargonate, caprate, undecylate, laurate, malonic acid, caproic acid, enanthic acid, caprylic acid, pelargonic acid, capric acid, undecylic acid, lauric acid, alkyl derivatives thereof, isomers thereof, or salts thereof.

The A and X groups in the polyamidoamines or polyamidoaminates can vary depending on the one or more reagents used to complex the polyamidoamines. Therefore, the values of y and z can also vary relative to the particular reagent combined with the polyamidoamine. For example, acetic acid is a monoprotic acid that provides one proton, e.g., H⁺, and one conjugate base, e.g., AcO⁻, while oxalic acid is a diprotic acid that provides two protons, e.g., 2H⁺, and one conjugate base, e.g., C₂O₄²⁻. In various compositions of the polyamidoamines or polyamidoaminates, y and z can be equal or substantially to each other, y can be greater than z, or z can be greater than y.

In some examples of the polyamidoamines or polyamidoaminates, y and/or z can be integers, such as 1, 2, 3, 4, 5, 6, 7, 8, 9, or 10. Alternatively, in other examples of the polyamidoamines or polyamidoaminates, y and/or z can be non-integers or fractions which can indicate a mixture of molecules which are partially cationic and/or anionic functionalized. Each y and z can independently be about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 5.5, about 6, about 6.5, about 7, about 7.5, about 8, about 8.5, about 9, about 9.5, about 10, or greater. In some examples, each y and z can independently be about 0.1 to about 8, about 0.5 to about 8, about 1 to about 8, about 1 to about 4. In other examples, each y and z can independently be 1 to 8, 1 to 7, 1 to 6, 1 to 5, 1 to 4, 1 to 3, or 1 to 2.

In one or more examples, illustrative polyamidoaminates can be or include one or more polyethylene polyamidoaminates having the chemical formula (K):

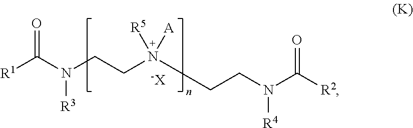

where R¹, R², R³, R⁴, each R⁵, and n are defined as above for the chemical formula (H), and where A and X are defined as above for the chemical formula (J). In some examples, A can be a hydrogen or a saturated or unsaturated, substituted or unsubstituted, linear or branched, cyclic, heterocyclic, or aromatic hydrocarbyl group.

In some illustrative polyethylene polyamidoaminates having chemical formula (K), R¹ and R² can independently be a saturated or unsaturated, substituted or unsubstituted, linear or branched, cyclic, heterocyclic, or aromatic hydrocarbyl group, R³, R⁴, and each R⁵ can independently be hydrogen or a saturated or unsaturated, substituted or unsubstituted, linear or branched, cyclic, heterocyclic, or aromatic hydrocarbyl group, n can be an integer of 1 to 8, A can be hydrogen, one or more alkali metals, one or more alkaline earth metals, ammonium, alkylammonium compounds, or one or more other cationic species, and X can be one or more conjugate bases, halides, e.g., F, Cl, Br, or I anions, hydroxide, or other anionic species.

In some examples, R¹ and R² can be an alkyl, an alkenyl, an alkynyl, an aryl, an alkoxyl, a carboxylic acid, an amino, an amido, a saturated and/or unsaturated fatty acid group, isomers thereof, combinations thereof, or mixtures thereof, R³, R⁴, and each R⁵ can be hydrogen, n can be 1, 2, 3, 4, or 5, A can be hydrogen, and X can be or include one or more conjugate bases which include acetate, glycolate, lactate, pyruvate, formate, propionate, butyrate, valerate, oxalate, alkyl derivatives thereof, isomers thereof, or mixtures thereof.

For example, R³, R⁴, and each R⁵ can all be hydrogen in the chemical formula (K), and the polyamidoaminates can be or include one or more polyethylene polyamidoaminates having the chemical formula (L):

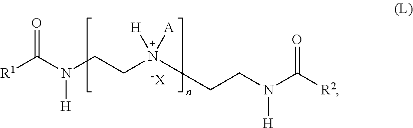

where R¹, R², n, A, and X are defined as above for the chemical formula (K).

In one or more examples, A can be hydrogen and X can be acetate in the chemical formula (L), and the polyethylene polyamidoaminates can be or include one or more polyethylene polyamidoamine acetates having the chemical formulas (M) and (N):

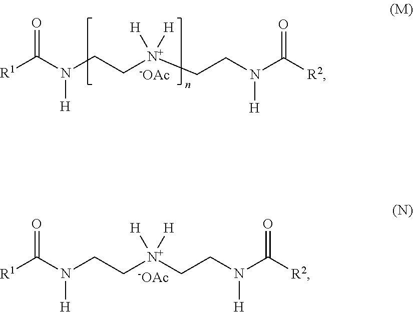

where R¹, R², and n are defined as above for the chemical formula (K).

In some examples, n can be 1 for the polyethylene polyamidoamine acetates having the chemical formula (M) to provide illustrative diethylene polyamidoamine acetates that have the chemical formula (N). In other illustrative polyethylene polyamidoamine acetates having the chemical formula (M), n can be 2, 3, 4, 5, or greater, for example, triethylene polyamidoamine acetate (n=2), tetraethylene polyamidoamine acetate (n=3), or pentaethylene polyamidoamine acetate (n=4).

In one or more examples, the polyamidoamines can be made, formed, synthesized, or otherwise produced by reacting one or more polyamines and one or more fatty acids or other carboxylic acids. The polyamidoamines can be produced by combining and reacting greater than one molar equivalent of the fatty acids with one molar equivalent of the polyamines. In some examples, the polyamidoamines can be produced by combining and reacting greater than 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.5, or about 5 molar equivalents of the fatty acid with one molar equivalent of the polyamine. Processes that can be used to produce the polyamidoamines from polyamine and fatty acid are discussed and described in, for example, U.S. Pat. Nos. 2,857,331; 2,927,692; and 3,166,548.

The one or more polyamines and one or more fatty acids or other carboxylic acids can be reacted to form the polyamidoamines at a temperature of about 0° C., about 10° C., about 20° C., about 25° C., about 30° C., about 40° C., about 50° C., about 60° C., about 70° C., about 80° C., about 90° C., about 100° C., about 110° C., about 120° C., about 130° C., about 140° C., about 150° C., about 160° C., about 170° C., about 180° C., about 190° C., about 200° C., about 250° C., or about 300° C. For example, the one or more polyamines and one or more fatty acids or other carboxylic acids can be reacted to form the polyamidoamines at a temperature of about 0° C. to about 300° C., about 10° C. to about 250° C., about 20° C. to about 225° C., about 20° C. to about 200° C., about 20° C. to about 190° C., about 20° C. to about 180° C., about 20° C. to about 175° C., about 20° C. to about 165° C., about 20° C. to about 150° C., about 50° C. to about 225° C., about 50° C. to about 500° C., about 50° C. to about 190° C., about 50° C. to about 180° C., about 50° C. to about 175° C., about 50° C. to about 165° C., about 50° C. to about 150° C., about 100° C. to about 225° C., about 100° C. to about 1000° C., about 100° C. to about 190° C., about 100° C. to about 180° C., about 100° C. to about 175° C., about 100° C. to about 165° C., about 100° C. to about 150° C., about 120° C. to about 225° C., about 120° C. to about 1200° C., about 120° C. to about 190° C., about 120° C. to about 180° C., about 120° C. to about 175° C., about 120° C. to about 165° C., or about 120° C. to about 150° C. The one or more polyamines and one or more fatty acids or other carboxylic acids can be reacted to form the polyamidoamines for about 10 min to about 24 hr, about 0.5 hr to about 12 hr, about 0.75 hr to about 10 hr, about 1 hr to about 5 hr, about 2 hr to about 4 hr, or about 3 hr.

In one or more examples, the composition can include one or more polyamidoamines that can be derived, formed, or otherwise produced from one or more polyamines or polyamine sources. The one or more polyamines or polyamine sources can be reacted with one or more fatty acids to produce or otherwise form the one or more polyamidoamines. Illustrative polyamines can include, but are not limited to, dimethylenetriamine, trimethylenetetramine, tetramethylenepentamine, pentamethylenehexamine, diethylenetriamine (DETA), triethylenetetramine (TETA), tetraethylenepentamine (TEPA), pentaethylenehexamine (PEHA), dipropylenetriamine, tripropylenetetramine, tetrapropylenepentamine, pentapropylenehexamine, dibutylenetriamine, tributylenetetramine, tetrabutylenepentamine, pentabutylenehexamine, aminoethylpiperazine, dipropylenetriamine, spermine, spermidine, heavy polyamine X (HPA X), tallow amines, isomers thereof, salts thereof, complexes thereof, adducts thereof, or any mixture thereof. In some examples, the polyamine can be or include a mixture of linear, branched, and/or cyclic ethyleneamines and/or other alkyleneamines, polyethylene polyamines, pentaethylenehexamine mixtures, tetraethylenepentamine mixtures, triethylenetetramine mixtures, isomers thereof, salts thereof, or any mixture thereof. For example, such polyamine can be or include heavy polyamine X (HPA X), commercially available from Dow Chemical Company, and can have components that contain six or more nitrogen atoms per molecule. Polyamine sources can be or include salts, adducts, complexes, or other forms of polyamines or other compounds which provide a source of polyamines which can be used to make polyamidoamines. Polyamine sources can produce polyamines, for example, but not limited to, upon heating, adjusting the pH or concentration, or reacting with other reagents or compounds.

In one or more examples, the composition can include one or more polyamidoamines that can be derived, formed, or otherwise produced, in part, from one or more fatty acids or fatty acid sources. The one or more fatty acids or fatty acid sources can be reacted with one or more polyamines to produce or otherwise form the one or more polyamidoamines. Illustrative fatty acids or fatty acid sources can be or include one or more fatty acids, TOFA, rosin acids, CTO, DTO, tall oil pitches, portions thereof, fractions thereof, or any mixture thereof. In some specific examples, the fatty acids or fatty acid sources can be or include TOFA, lauric acid, stearic acid, isostearic acid, naphthenic acid, oleic acid, linoleic acid, linolenic acid, palmitic acid, coconut oil fatty acid, isomers thereof, or any mixture thereof.

In one or more examples, the fatty acids or fatty acid sources can be or include coconut oil fatty acids. Illustrative coconut oil fatty acids can include lauric acid, myristic acid, palmitic acid, capric acid, caprylic acid, oleic acid, stearic acid, palmitoleic acid, linoleic acid, caproic acid, arachidic acid, one or more other fatty acids, isomers thereof, or any mixture thereof. The composition can include one or more polyamidoamines that can be derived, formed, or otherwise produced from at least 6, at least 7, at least 8, at least 9, at least 10, or at least 11 fatty acids selected from lauric acid, myristic acid, palmitic acid, capric acid, caprylic acid, oleic acid, stearic acid, palmitoleic acid, linoleic acid, caproic acid, arachidic acid, one or more other fatty acids, isomers thereof, or any mixture thereof. For example, the one or more polyamidoamines can be derived, formed, or otherwise produced from about 6 to about 10, about 7 to about 10, about 8 to about 10, about 6 to about 11, about 7 to about 11, about 8 to about 11, about 9 to about 11, or about 10 to about 11 fatty acids selected from lauric acid, myristic acid, palmitic acid, capric acid, caprylic acid, oleic acid, stearic acid, palmitoleic acid, linoleic acid, caproic acid, arachidic acid, one or more other fatty acids, isomers thereof, or any mixture thereof. In some examples, the coconut oil fatty acids can include about 40 wt % to about 55 wt % of lauric acid, about 10 wt % to about 25 wt % of myristic acid, about 5 wt % to about 15 wt % of palmitic acid, about 4 wt % to about 15 wt % of capric acid, about 3 wt % to about 12 wt % of caprylic acid, about 3 wt % to about 12 wt % of oleic acid, about 0.5 wt % to about 5 wt % of stearic acid, about 0.01 wt % to about 5 wt % of palmitoleic acid, about 0.01 wt % to about 3 wt % of linoleic acid, about 0.01 wt % to about 2.5 wt % of caproic acid, about 0.01 wt % to about 2.5 wt % of arachidic acid, isomers thereof, or any mixture thereof. In other examples, the coconut oil fatty acids can include about 44 wt % to about 52 wt % of lauric acid, about 13 wt % to about 19 wt % of myristic acid, about 8 wt % to about 11 wt % of palmitic acid, about 6 wt % to about 10 wt % of capric acid, about 5 wt % to about 9 wt % of caprylic acid, about 5 wt % to about 8 wt % of oleic acid, about 1 wt % to about 3 wt % of stearic acid, about 0.01 wt % to about 2.5 wt % of palmitoleic acid, about 0.01 wt % to about 1 wt % of linoleic acid, about 0.01 wt % to about 0.8 wt % of caproic acid, about 0.01 wt % to about 0.5 wt % of arachidic acid, isomers thereof, or any mixture thereof.

In one example, CTO can be made or produced as an acidified byproduct in the kraft or sulfate processing of wood. Crude tall oil, prior to refining, can include a mixture of rosin acids, fatty acids, sterols, high-molecular weight alcohols, and other alkyl chain materials. The components of CTO can depend on a variety of factors, such as the particular species of the wood being processed (wood type), the geographical location of the wood source, the age of the wood, the particular season that the wood is harvested, and others. Thus, depending on the particular source, CTO can contain about 20 wt % to about 75 wt % of fatty acids, e.g., about 30 wt % to about 60 wt % of fatty acids, about 20 wt % to about 65 wt % of rosin acids, e.g., about 30 wt % to about 60 wt % of rosin acids, and the balance being neutral and non-saponifiable components. In some examples, the CTO can include at least 8 wt % or about 10 wt % of neutral materials or non-saponifiable components.

Distillation of CTO can be used to recover a mixture of fatty acids, referred to as DTO or DTO fraction, which can have about 16 carbon atoms to about 20 carbon atoms. In some examples, these fatty acids can be included with the polyamines to produce or otherwise form the polyamidoamines. Fatty acids found in tall oils can include, but are not limited to, oleic acid, linoleic acid, stearic acid, and palmitic acid. Rosin acids found in tall oils, include, but are not limited to, abietic acid, dehydroabietic acid, isopimaric acid, and pimaric acid.

The DTO fraction can have a fatty acids and/or esters of fatty acids concentration of about 55 wt %, about 60 wt %, or about 65 wt % to about 85 wt %, about 90 wt %, or about 95 wt %. The DTO fraction can have a rosin acids or rosins concentration of about 5 wt %, about 10 wt %, or about 15 wt % to about 30 wt %, about 35 wt %, or about 40 wt %. The DTO fraction can have a neutrals concentration of about 0.1 wt %, about 1 wt %, or about 1.5 wt % to about 2 wt %, about 3.5 wt %, or about 5 wt %. The DTO fraction can have an acid value of about 20, about 25, or about 30 to about 40, about 45, or about 50. The DTO fraction can have a viscosity (centipoise at 85° C.) of about 10 cP, about 20 cP, about 30 cP, or about 40 cP to about 100 cP, about 120 cP, about 135 cP, or about 150 cP. The distilled tall oil can have a density of about 840 g/L, about 860 g/L, or about 880 g/L to about 900 g/L, about 920 g/L, or about 935 g/L. The DTO fraction can have a saponification number of about 180, about 185, or about 190 to about 200, about 205, or about 210. The DTO fraction can have an iodine value of about 115, about 117, or about 120 to about 130, about 135, or about 140.

The rosin acids derived from CTO are also an intermediate fraction that can be produced from the distillation of CTO. The tall oil rosin can have a concentration of rosin acids of about 80 wt %, about 85 wt %, or about 90 wt % to about 93 wt %, about 95 wt %, or about 99 wt %. The tall oil rosin can have a concentration of abietic acid of about 35 wt %, about 40 wt %, or about 43 wt % to about 50 wt %, about 55 wt %, or about 60 wt %. The tall oil rosin can have a concentration of dehydroabietic acid of about 10 wt %, about 13 wt %, or about 15 wt % to about 20 wt %, about 23 wt %, or about 25 wt %. The tall oil rosin can have a concentration of isopimaric acid of about 10 wt % or less, about 8 wt % or less, about 5 wt % or less, or about 3 wt % or less. The tall oil rosin can have a concentration of pimaric acid of about 10 wt % or less, about 8 wt % or less, about 5 wt % or less, or about 3 wt % or less. The tall oil rosin can have a fatty acids concentration of about 0.5 wt %, about 1 wt %, or about 2 wt % to about 3 wt %, about 5 wt %, or about 10 wt %. The tall oil rosin can have a concentration of neutral materials of about 0.5 wt %, about 1 wt %, or about 2 wt % to about 3 wt %, about 5 wt %, or about 10 wt %. The tall oil rosin can have a density of about 960 g/L, about 970 g/L, or about 980 g/L to about 1,000 g/L, about 1,010 g/L, or about 1,020 g/L. The tall oil rosin can have an acid value of about 150, about 160, or about 165 to about 170, about 175, or about 180.

Representative tall oil products, which can be fatty acid sources used to form the polyamidoamines, can be or include, but are not limited to, saturated and unsaturated fatty acids in the C₁₆-C₁₈ range, as well as minor amounts of rosin acids, and can include XTOL® 100, XTOL® 300, and XTOL® 304, XTOL® 520, and LYTOR® 100, all of which are commercially available from Georgia-Pacific Chemicals LLC, Atlanta, Ga. XTOL® 100 includes about 1.6 wt % of palmitic acid, about 2.5 wt % of stearic acid, about 37.9 wt % of oleic acid, about 26.3 wt % of linoleic acid, about 0.3 wt % of linolenic acid, about 2.9 wt % of linoleic isomers, about 0.2 wt % of arachidic acid, about 3.6 wt % eicosatrienoic acid, about 1.4 wt % of pimaric acid, <0.16 wt % of sandarocopimaric, <0.16 wt % of isopimaric acid, <0.16 wt % of dehydroabietic acid, about 0.2 wt % of abietic acid, with the balance being neutrals and high molecular weight species. LYTOR® 100 includes <0.16 wt % of palmitic acid, <0.16 wt % of stearic acid, about 0.2 wt % of oleic acid, about 0.2 wt % of arachidic acid, about 0.2 wt % eicosatrienoic acid, about 2.2 wt % of pimaric acid, about 0.6 wt % of sandarocopimaric, about 8.5 wt % of palustric acid, about 1.6 wt % of levopimaric acid, about 2.8 wt % of isopimaric acid, about 15.3 wt % of dehydroabietic acid, about 51.4 wt % of abietic acid, about 2.4 wt % of neoabietic acid, with the balance being neutrals and high molecular weight species. XTOL® 520 DTO includes about 0.2 wt % of palmitic acid, about 3.3 wt % of stearic acid, about 37.9 wt % of oleic acid, about 26.3 wt % of linoleic acid, about 0.3 wt % of linolenic acid, about 2.9 wt % of linoleic isomers, about 0.2 wt % of arachidic acid, about 3.6 wt % eicosatrienoic acid, about 1.4 wt % of pimaric acid, <0.16 wt % wt % of sandarocopimaric, <0.16 wt % of isopimaric acid, <0.16 wt % of dehydroabietic acid, about 0.2 wt % of abietic acid, with the balance being neutrals and high molecular weight species. Such tall oil products can be used in the reaction with the polyamine or a mixture of polyamines. Other fatty acids and mixtures of fatty acids, including oxidized and/or dimerized tall oil, such those discussed below can also be employed.

In one or more examples, illustrative fatty acid sources can be or include a fatty acid, a mixture of fatty acids, a fatty acid ester, a mixture of fatty acid esters, or a mixture of one or more fatty acids and one or more fatty acid esters. The fatty acid sources or fatty acids can be combined with the tall oils and one or more polyamines, and subsequently reacted to produce or otherwise form the one or more polyamidoamines. In other examples, the fatty acid sources or fatty acids can be used instead of the tall oils, therefore, the fatty acids can be reacted with one or more polyamines to produce or otherwise form the one or more polyamidoamines. Illustrative fatty acid sources or fatty acids can be or include, but are not limited to, oleic acid, lauric acid, linoleic acid, linolenic acid, palmitic acid, stearic acid, isostearic acid, ricinoleic acid, myristic acid, arachidic acid, behenic acid, capric acid, caprylic acid, caproic acid, palmitoleic acid, isomers thereof, or any mixture thereof.

In some examples, fatty acid sources or fatty acids which can be reacted with one or more polyamines to produce or otherwise form the one or more polyamidoamines can include fatty acids from various plant and/or vegetable oil sources. Illustrative plant or vegetable oils that can be used as the fatty acids can include, but are not limited to, safflower oil, grapeseed oil, sunflower oil, walnut oil, soybean oil, cottonseed oil, coconut oil, corn oil, olive oil, palm oil, palm olein, peanut oil, rapeseed oil, canola oil, sesame oil, hazelnut oil, almond oil, beech nut oil, cashew oil, macadamia oil, mongongo nut oil, pecan oil, pine nut oil, pistachio oil, grapefruit seed oil, lemon oil, orange oil, watermelon seed oil, bitter gourd oil, buffalo gourd oil, butternut squash seed oil, egusi seed oil, pumpkin seed oil, borage seed oil, blackcurrant seed oil, evening primrose oil, acai oil, black seed oil, flaxseed oil, carob pod oil, amaranth oil, apricot oil, apple seed oil, argan oil, avocado oil, babassu oil, ben oil, borneo tallow nut oil, cape chestnut, algaroba oil, cocoa butter, cocklebur oil, poppyseed oil, cohune oil, coriander seed oil, date seed oil, dika oil, false flax oil, hemp oil, kapok seed oil, kenaf seed oil, lallemantia oil, mafura oil, manila oil, meadowfoam seed oil, mustard oil, okra seed oil, papaya seed oil, perilla seed oil, persimmon seed oil, pequi oil, pili nut oil, pomegranate seed oil, prune kernel oil, quinoa oil, queef oil, ramtil oil, rice bran oil, royle oil, shea nut oil, sacha inchi oil, sapote oil, seje oil, taramira oil, tea seed oil, thistle oil, tigernut oil, tobacco seed oil, tomato seed oil, wheat germ oil, castor oil, colza oil, flax oil, radish oil, salicornia oil, tung oil, honge oil, jatropha oil, jojoba oil, nahor oil, paradise oil, petroleum nut oil, dammar oil, linseed oil, stillingia oil, vernonia oil, amur cork tree fruit oil, artichoke oil, balanos oil, bladderpod oil, brucea javanica oil, burdock oil, candlenut oil, carrot seed oil, chaulmoogra oil, crambe oil, croton oil, cuphea oil, honesty oil, mango oil, neem oil, oojon oil, rose hip seed oil, rubber seed oil, sea buckthorn oil, sea rocket seed oil, snowball seed oil, tall oil, tamanu oil, tonka bean oil, ucuhuba seed oil, or any mixture thereof. Illustrative animal fats or oils that can be used as the fatty acids can include, but are not limited to, fatty acids from animal sources, such as cows, pigs, lambs, chickens, turkeys, ducks, geese, and other animals, as well as dairy products such as milk, butter, or cheese. Illustrative fatty acids from animal sources can include palmitic acid, stearic acid, myristic acid, oleic acid, palmitoleic acid, linoleic acid, or any mixture thereof.

If the fatty acid source includes two or more fatty acids, each fatty acid can be present in the same amount or different amounts with respect to one another. For example, a first fatty acid can be present with respect to another or “second” fatty acid contained therein in a weight ratio of about 10,000:1, about 9,000:1, about 8,000:1, about 7,000:1, about 6,000:1, about 5,000:1, about 4,000:1, about 3,000:1, about 2,000:1, about 1,000:1, about 900:1, about 800:1, about 700:1, about 600:1, about 500:1, about 400:1, about 300:1, about 200:1, about 150:1, about 100:1, about 99:1, about 90:10, about 80:20, about 70:30, about 60:40, about 50:50, about 40:60, about 30:70, about 20:80, about 10:90, about 1:99, about 1:100, about 1:150, about 1:200, about 1:300, about 1:400, about 1:500, about 1:600, about 1:700, about 1:800, about 1:900, about 1,000, about 2,000, about 1:3,000, about 1:4,000, about 1:5,000, about 1:6,000, about 1:7,000, about 1:8,000, about 1:9,000, or about 1:10,000. Similarly, if three or more fatty acids are mixed, the three or more fatty acids can be present in any ratio. Therefore, the two or more fatty acids can be reacted with one or more polyamines to produce or otherwise form polyamidoamines with different R¹ and R² groups in any of the chemical formulas (A)-(M).

In some examples, one or more organic acid sources or organic acids can be combined with one or more fatty acid sources or fatty acids and one or more polyamines, and subsequently reacted to produce or otherwise form the one or more polyamidoamines. In other examples, organic acid sources or organic acids can be used instead of the fatty acid sources or fatty acids, therefore, the organic acid sources or organic acids can be reacted with one or more polyamines to produce or otherwise form the one or more polyamidoamines. Illustrative organic acid sources or organic acids can include, but are not limited to, glycolic acid, lactic acid, pyruvic acid, formic acid, acetic acid, propionic acid, butyric acid, valeric acid, oxalic acid, malonic acid, caproic acid, enanthic acid, caprylic acid, pelargonic acid, capric acid, undecylic acid, lauric acid, isomers thereof, hydrates thereof, salts thereof, complexes thereof, adducts thereof, or any mixture thereof.

In some specific examples, the polyamines that can be reacted with the fatty acids to produce the polyamidoamines can be or include diethylenetriamine, triethylenetetramine, tetraethylenepentamine, or a mixture thereof, and the fatty acids that can be reacted with the polyamines to produce the polyamidoamines can be or include tall oil fatty acids, lauric acid, stearic acid, isostearic acid, naphthenic acid, isomers thereof, or any mixture thereof.

The polyamidoamine can have a total amine value (TAV) of about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, or about 300, based on mg of KOH per g of polyamidoamine. In some examples, the polyamidoamine can have a TAV of about 50 to about 300, about 50 to about 200, about 50 to about 100, about 80 to about 120, about 180 to about 300, about 200 to about 300, about 220 to about 280, about 230 to about 270, about 240 to about 260, or about 245 to about 255, based on mg of KOH per g of polyamidoamine.

In one or more examples, a critical micelle concentration (CMG) of a polyamidoamine (having any one of the chemical formulas (A)-(M)) can be increased where R¹ and R² are or include different hydrocarbyl groups, relative to a polyamidoamine, having the same chemical formula except where R¹ and R² are or include the same hydrocarbyl group. Disorder in chemical structure, such as branching or irregularities in the R¹ and R² hydrocarbyl groups, can inhibit formation of micelles in the aqueous solutions or slurries. In some examples, preventing or minimizing micelle formation can provide more available polyamidoamine for adhering to mineral particles in the aqueous solutions or slurries. In other aspects, the irregularities in structure can also provide lower melting points for the polyamidoamine and/or the composition, which in turn can be increase the utility of the polyamidoamine or the composition in cold-weather applications.

In other examples, the composition can include a polyamidoamine that can have varying carbon chain lengths in the R¹ and R² hydrocarbyl groups can be prepared to have a desired collecting power based on mineral particle size in the aqueous solutions or slurries. In some examples, if the R¹ and R² hydrocarbyl groups include long carbon chains, such as a C18 to C24 chain, then the polyamidoamine can favor collecting particles having a particle size of greater than 150 μm, such as greater than 150 μm to about 750 μm. Alternatively, in other examples, if the R¹ and R² hydrocarbyl groups include short carbon chains, such as a C6 to C12 chain, then the polyamidoamine can favor collecting particles having a particle size of less than 75 μm, such as less than 75 μm to about 5 μm. In some examples, diamidoamines, triamidoamines, and other polyamidoamines having intermediate sized carbon chain lengths, such as a C6 to C24 chain, can be produced or otherwise formed from mixed fatty acids with varying carbon chain lengths, then the polyamidoamine can favor collecting particles having a particle size of about 75 μm to about 150 μm.

In one or more examples, a hydrophilic-lipophilic balance (HLB) value of the polyamidoamine can be tuned or otherwise selected, at least in part, by varying the carbon chain lengths of the R¹ and R² hydrocarbyl groups. Polyamidoamines having an intermediate HLB value can be produced which would not be obtained from synthesis with a single fatty acid. The HLB value of the polyamidoamine can be determined by the Davies' Method that assigns a value to different functional groups based on polarity and also uses the following equation:

HLB=7+mHₕ+nH₁,

where “Hₕ” is the value assigned to each specific hydrophilic functional group, “m” is the numerical amount of the specified hydrophilic functional groups, “H₁” is the value assigned to each specific lipophilic group, and “n” is the numerical amount of the specified lipophilic groups. For example, Hₕ can have an amine value of 10 and an amide value of 4, and H₁ can have a CHₙ value of −0.475. The HLB value and the equation for determining the HLB value by the Davies' Method are discussed and described in, for example, Davies, J. T., “A Quantitative Kinetic Theory of Emulsion Type, I. Physical Chemistry of the Emulsifying Agent,” Gas/Liquid and Liquid/Liquid Interfaces, Proceedings of the 2^{nd} International Congress Surface Activity, Butterworths, London, pgs. 426-438, 1957.

In one or more examples, the polyamidoamines can generally have an HLB of about 2, about 5, about 8, about 10, about 12, about 15, about 18, about 20, about 25, about 30, about 35, about 40, about 45, or about 50, based on the Davies' Method for hydrophilic-lipophilic balance. For example, the polyamidoamines can generally have an HLB of about 2 to about 50, about 5 to about 50, about 5 to about 20, about 5 to about 15, about 10 to about 25, about 10 to about 20, about 20 to about 35, about 20 to about 30, about 25 to about 35, about 25 to about 30, or about 30 to about 35, based on the Davies' Method for hydrophilic-lipophilic balance.

In some examples, the polyamidoamines can be or include one or more amidoamines having chemical formula (A), where n can be 2 and the polyamidoamine can have an HLB of about 7.5 to about 12, n can be 3 and the polyamidoamine can have an HLB of about 16.5 to about 21, or n can be 4 and the polyamidoamine can have an HLB of about 25.5 to about 30, based on the Davies' Method for hydrophilic-lipophilic balance. In other examples, the polyamidoamines can be or include one or more amidoamines having chemical formula (A), where n can be 2 and the polyamidoamine can have an HLB of about 8.5 to about 11, n can be 3 and the polyamidoamine can have an HLB of about 17.5 to about 20, or n can be 4 and the polyamidoamine can have an HLB of about 26 to about 29, based on the Davies' Method for hydrophilic-lipophilic balance. In other examples, the polyamidoamines can be or include one or more amidoamines having chemical formula (A), where n can be 2 and the polyamidoamine can have an HLB of about 9 to about 10.5, n can be 3 and the polyamidoamine can have an HLB of about 18 to about 19.5, or n can be 4 and the polyamidoamine can have an HLB of about 26.5 to about 28.5 or about 27 to about 28, based on the Davies' Method for hydrophilic-lipophilic balance.

In some examples, a mixture of two, three, or more polyamidoamines having any one of the chemical formulas (A)-(D) can have an HLB of about 7.5 to about 30 and can include one or more polyamidoamines having any one of the chemical formulas (A)-(D) where n is 2, one or more polyamidoamines having any one of the chemical formulas (A)-(D) where n is 3, one or more polyamidoamines having any one of the chemical formulas (A)-(D) where n is 4 or 5, or any mixture thereof. In other examples, a mixture of two, three, or more polyethylenepolyamidoamines can have an HLB of about 7.5 to about 30 and can include one or more polyethylenepolyamidoamines having the chemical formula (E), one or more polyethylenepolyamidoamines having the chemical formula (F), one or more polyethylenepolyamidoamines having the chemical formula (G), or any mixture thereof.

In one or more examples, the polyamidoamines can be or include a mixture of three or more polyamidoamines having any one of the chemical formulas (A)-(M), where the mixture can include at least a first diamidoamine, a second diamidoamine, and a third diamidoamine. The first diamidoamine can have R¹ and R² as the same hydrocarbyl group, a second diamidoamine can have R¹ and R² as the same hydrocarbyl group, but different hydrocarbyl groups as the first diamidoamine, and the third diamidoamine can have R¹ and R² as different hydrocarbyl groups, such that the R¹ hydrocarbyl group in the third diamidoamine is the same as the R¹ and R² hydrocarbyl groups in the first diamidoamine and the R² hydrocarbyl group in the third diamidoamine is the same as the R¹ and R² hydrocarbyl groups in the second diamidoamine. For example, the R¹ and R² hydrocarbyl groups in the first diamidoamine and the R¹ hydrocarbyl group in the third diamidoamine can be same hydrocarbyl group, and the R¹ and R² hydrocarbyl groups in the second diamidoamine and the R² hydrocarbyl group in the third diamidoamine can be same hydrocarbyl group, but different than the R¹ and R² hydrocarbyl groups in the first diamidoamine and the R¹ hydrocarbyl group in the third diamidoamine.

In one or more examples, the polyamidoamines can be or include a mixture of two, three, or more polyamidoamines having any one of the chemical formulas (A)-(M), where the mixture can include polyamidoamines of different amounts of amido groups and/or amine groups. For example, the mixture of polyamidoamines can include a first polyamidoamine having any one of the chemical formulas (A)-(D) where n is 2 and a second polyamidoamine having any one of the chemical formulas (A)-(D) where n is 3, 4, or 5. In another example, the mixture of polyamidoamines can include a first polyamidoamine having any one of the chemical formulas (A)-(D) where n is 2, a second polyamidoamine having any one of the chemical formulas (A)-(D) where n is 3, and a third polyamidoamine having any one of the chemical formulas (A)-(D) where n is 4 or 5. In some examples, the mixture of polyamidoamines can be or include a mixture of polyethylenepolyamidoamines, such as, but is not limited to, the polyethylenepolyamidoamines having the chemical formulas (E), (F), and (G). For example, the mixture of polyethylenepolyamidoamines can include a first polyethylenepolyamidoamine having the chemical formula (E) and a second polyethylenepolyamidoamine having the chemical formula (F) or (G). In another example, the mixture of polyethylenepolyamidoamines can include a first polyethylenepolyamidoamine having the chemical formula (E), a second polyethylenepolyamidoamine having the chemical formula (F), and a third polyethylenepolyamidoamine having the chemical formula (G).

In one or more examples, an aqueous mixture can include one or more ores, one or more polyamidoamines, optionally acetic acid and/or other organic acid, and water, where the polyamidoamines can be or include one or more amidoamines having any one of the chemical formulas (A)-(M). In some examples, an aqueous mixture of an ore can include one or more phosphorous ores, one or more polyamidoamines, optionally acetic acid and/or other organic acid, and water, where the polyamidoamines can be or include one or more amidoamines having any one of the chemical formulas (A)-(M). In some examples, an aqueous mixture of an ore can include one or more phosphorous ores, one or more polyamidoamines, optionally acetic acid and/or other organic acid, and water, where the polyamidoamines can be or include one or more amidoamines having any one of the chemical formulas (A)-(M).

In some examples, the polyamidoamines can be or include one or more amidoamines having chemical formula (A), where R¹ and R² can be different and can be selected from a saturated or unsaturated, substituted or unsubstituted, linear or branched, cyclic, heterocyclic, or aromatic hydrocarbyl group, R³ and R⁴ can independently be hydrogen or a saturated or unsaturated, substituted or unsubstituted, linear or branched, cyclic, heterocyclic, or aromatic hydrocarbyl group, each m can be an integer of 1 to 5, and n can be an integer of 2 to 8. In other examples, the polyamidoamines can be or include one or more amidoamines having the chemical formula (B), where R¹ and R² can be different and can be selected from C₉H₁₉, C₉H₁₇, C₉H₁₅, C₉H₁₃, C₁₁H₂₃, C₁₁H₂₁, C₁₅H₃₃, C₁₅H₃₁, C₁₅H₂₉, C₁₇H₃₅, C₁₇H₃₃, C₁₇H₃₁, C₁₇H₂₉, C₁₉H₃₇, C₁₉H₃₅, C₁₉H₃₃, C₁₉H₃₁, or C₁₉H₂₉, and n can be an integer of 2, 3, or 4.

In one example, the polyamidoamine can be or include one or more products formed by reacting a polyamine and a fatty acid, where the polyamine can be or include diethylenetriamine, triethylenetetramine, tetraethylenepentamine, pentaethylenehexamine, or any mixture thereof, and the fatty acid can be or include tall oil fatty acids, coconut oil fatty acids, lauric acid, stearic acid, isostearic acid, naphthenic acid, oleic acid, linoleic acid, linolenic acid, palmitic acid, isomers thereof, or any mixture thereof. In some specific examples, the polyamine can be or include one or more diethylenetriamine, triethylenetetramine, tetraethylenepentamine, or any mixture thereof, and the fatty acid can be or include one or more tall oil fatty acids, lauric acid, stearic acid, isostearic acid, naphthenic acid, isomers thereof, or any mixture thereof. In other examples, a mixture of fatty acids can be used to make or form the polyamidoamines. The mixture of fatty acids can be or include at least 2, 3, 4, 5, 6, 7, 8, 9, or 10 fatty acids selected from lauric acid, myristic acid, palmitic acid, capric acid, caprylic acid, oleic acid, stearic acid, palmitoleic acid, linoleic acid, caproic acid, arachidic acid, isomers thereof, or any mixture thereof.

In another example, one or more organic acids can be combined with one or more polyamidoamines to make, form, or otherwise produce the composition and/or the aqueous mixture. In some examples, the one or more organic acids can be combined with the polyamidoamine and subsequently added with other components to make, form, or otherwise produce the composition and/or the aqueous mixture. In other examples, the one or more organic acids and the polyamidoamine can independently be combined with one or more components to make, form, or otherwise produce the composition and/or the aqueous mixture. When the one or more organic acids and the polyamidoamine are independently combined with one or more components, the organic acid and the polyamidoamine can be combined at the same time, the organic acid can be added before the polyamidoamine, or the polyamidoamine can be added before the organic acid. Illustrative organic acid sources or organic acids can include glycolic acid, lactic acid, pyruvic acid, formic acid, acetic acid, propionic acid, butyric acid, valeric acid, oxalic acid, malonic acid, caproic acid, enanthic acid, caprylic acid, pelargonic acid, capric acid, undecylic acid, lauric acid, isomers thereof, hydrates thereof, salts thereof, complexes thereof, adducts thereof, or any mixture thereof.

In some examples, one or more ores such as phosphorous containing materials can be purified by agitating, blending, mixing, or otherwise combining the ore, one or more polyamidoamines, and water to produce an aqueous mixture and recovering a purified product therefrom. In some example, one or more organic acids, e.g., acetic acid, can also be combined with the ore, the polyamidoamine, and water to produce the aqueous mixture. In some examples, any or all of the ore, the polyamidoamine, organic acid, and water can be combined with one another, in any order, to produce the aqueous mixture. In other examples, the ore, the polyamidoamines, the organic acid, and water can be separately combined with one another, in any order, to produce the aqueous mixture. In other examples, the polyamidoamine and the organic acid can be combined with one another to produce a first mixture and the ore and water can be combined to produce a second mixture and the first and second mixtures can be combined to produce the aqueous mixture. In some examples, the one or more polyamidoamines and organic acid can be combined with one another to produce the composition, and subsequently, the composition can be combined with the ore and water to produce the aqueous mixture. In other examples, the one or more polyamidoamines, organic acid, and water can be combined with one another to produce the composition, and subsequently, the composition can be added or combined with the ore material and if desired additional water to produce the aqueous mixture.

In some examples, a phosphorous containing material can be purified by combining one or more phosphorous ores, the polyamidoamine, an organic acid, and water to produce an aqueous mixture and collecting or recovering a phosphate material from the aqueous mixture. Generally, in some examples, silicates, silicon oxides, and/or other gangue materials can be floated away from the aqueous mixture or slurry providing the beneficiation or purification of the phosphate material. The polyamidoamines can be or include one or more amidoamines having any one of the chemical formulas (A)-(M). In some examples, the method can include the use of the amidoamines having any one of the chemical formulas (A)-(M), where R¹ and R² can be different and can be selected from a saturated or unsaturated, substituted or unsubstituted, linear or branched, cyclic, heterocyclic, or aromatic hydrocarbyl group. The organic acid can be or include acetic acid, e.g., glacial acetic acid.

In some examples, the purification or beneficiation of the ore such as a phosphorous ore or other phosphorous containing material can include the use of the amidoamines having the chemical formula (A), where R¹ and R² can be different and can be selected from a saturated or unsaturated, substituted or unsubstituted, linear or branched, cyclic, heterocyclic, or aromatic hydrocarbyl group, R³ and R⁴ can independently be hydrogen or a saturated or unsaturated, substituted or unsubstituted, linear or branched, cyclic, heterocyclic, or aromatic hydrocarbyl group, each m can be an integer of 1 to 5, and n can be an integer of 2 to 8. In some examples, the method can include the use of the amidoamines having the chemical formula (A), where R¹ and R² can be a C10 to C18 chain having 0 to 3 unsaturated bonds, R³ and R⁴ can be hydrogen, each m can be an integer of 2 or 3, and n can be an integer of 2, 3, or 4.

In some examples, the purification or beneficiation of the ore such as a phosphorous containing material can include the use of the amidoamines having the chemical formula (B), where R¹ and R² can be different and can be selected from a saturated or unsaturated, substituted or unsubstituted, linear or branched, cyclic, heterocyclic, or aromatic hydrocarbyl group, R³ and R⁴ can independently be hydrogen or a saturated or unsaturated, substituted or unsubstituted, linear or branched, cyclic, heterocyclic, or aromatic hydrocarbyl group, and n can be an integer of 2 to 8.

The polyamidoamines can be produced by reacting one or more polyamines and one or more fatty acids, where the polyamines can be or include diethylenetriamine, triethylenetetramine, tetraethylenepentamine, pentaethylenehexamine, or any mixture thereof, and the fatty acids can be or include tall oil fatty acids, lauric acid, stearic acid, isostearic acid, naphthenic acid, oleic acid, linoleic acid, linolenic acid, palmitic acid, isomers thereof, or any mixture thereof.

In some examples, the purification or beneficiation of a phosphorous containing material can include combining the organic acid, e.g., acetic acid, and the polyamidoamine to produce a cationic collector and combining the cationic collector and the phosphorous ore to produce the aqueous mixture. The acetic acid can be glacial acetic acid. The cationic collector can include about 10 wt % to about 60 wt % of the acetic acid and about 40 wt % to about 95 wt % of the polyamidoamine, based on the combined weight of the polyamidoamine and the acetic acid. The cationic collector can also include about 2 wt % to about 50 wt % of water, based on the combined weight of the polyamidoamine, the acetic acid, and the water. The composition can include about 10 wt % to about 60 wt % of acetic acid and about 40 wt % to about 95 wt % of the polyamidoamine, based on a combined weight of the polyamidoamine and the acetic acid. The composition can also include about 2 wt % to about 50 wt % of water, based on the combined weight of the polyamidoamine, the acetic acid, and the water.

The aqueous mixture can also be contacted with a gas, e.g., air. For example, the aqueous mixture can be by passing air bubbles or other gas bubbles through the aqueous mixture, mechanically stirring, e.g., impeller, paddle, and/or stirrer; shaking; directing sound waves, e.g., ultrasonic sound waves, into the aqueous mixture, or otherwise moving the aqueous mixture, or any combination thereof. The aqueous mixture can be an aqueous solution, slurry, suspension, dispersion, or the like.

The cationic collectors containing the polyamidoamines and the purification or beneficiation methods that use the cationic collectors can be used to recover, collect, or otherwise purify one or more materials from less pure mixtures, such as an ores. The cationic collectors can be used in froth flotation processes for the beneficiation of a wide variety of ores. The ore can generally be or include an aggregate of minerals and gangue from which one or more metals and/or oxides thereof can be separated or extracted. Illustrative ores that can be purified can be or include, but are not limited to, minerals, elements, and/or metals. Illustrative metals can be or include, but are not limited to, phosphorous, e.g., phosphate or other phosphorous oxides, iron, copper, aluminum, nickel, gold, silver, platinum, palladium, titanium, chromium, molybdenum, tungsten, manganese, magnesium, lead, zinc, potassium, e.g., potash, sodium, calcium, graphite, uranium, cerium, dysprosium, erbium, europium, gadolinium, holmium, lanthanum, lutetium, neodymium, praseodymium, promethium, samarium, scandium, terbium, thulium, ytterbium, yttrium, potash, feldspar, bauxite, other precious metals thereof, oxides thereof, ores thereof, or any mixture thereof. In one or more examples, the ore, such as a crude mineral ore to be beneficiated and produce a purified ore or material, can be or include, but is not limited to, a phosphorous ore, an iron ore, an aluminum ore, a potassium ore, a sodium ore, a calcium ore, potash, feldspar, bauxite, any mixture thereof. The raw materials to be purified and recovered generally contains or includes gangue. The gangue can be or include one or more silicates, sand, quartz, clay, rocks, other materials, or any mixture thereof. The cationic collectors can be selective toward the gangue, and especially selective toward silicates, sand, quartz, and other silicon oxide materials.

In one or more examples, the cationic collector containing one or more polyamidoamines and organic acid, e.g., acetic acid, can be used in froth flotation processes for the beneficiation of phosphorous containing materials, such as phosphate. The phosphorous or phosphate containing ores, e.g., rocks, minerals, or other materials, as well as the recovered or collected purified ore can include one or more tribasic phosphate salts. The tribasic phosphate salts can include alkaline earth metals, alkali metals, adducts thereof, complexed salts thereof, hydrates thereof, or mixtures thereof. For example, the phosphorous ore or the phosphate material can include calcium phosphate.

In one or more examples, the amount of the polyamidoamine in the composition or the cationic collector can be about 20 wt %, about 30 wt %, about 40 wt %, about 50 wt %, about 55 wt %, about 60 wt %, about 65 wt %, about 70 wt %, about 75 wt %, about 80 wt %, about 85 wt %, about 90 wt %, about 91 wt %, about 92 wt %, about 93 wt %, about 94 wt %, about 95 wt %, about 96 wt %, about 97 wt %, about 97.5 wt %, about 98 wt %, about 98.5 wt %, about 99 wt %, about 99.3 wt %, or about 99.5 wt %, based on the combined weight of the polyamidoamine and the organic acid, e.g., acetic acid. In some examples, the amount of the polyamidoamine in the composition or the cationic collector can be about 20 wt % to about 99 wt %, about 30 wt % to about 98 wt %, about 30 wt % to about 95 wt %, about 30 wt % to about 90 wt %, about 40 wt % to about 99 wt %, about 40 wt % to about 95 wt %, about 50 wt % to about 98 wt %, or about 50 wt % to about 95 wt %, based on the combined weight of the polyamidoamine and the organic acid. In one or more examples, the organic acid can be or include acetic acid in the cationic collector.

The amount of the organic acid, e.g., acetic acid in the composition or the cationic collector can be about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, or about 90 wt %, based on the combined weight of the polyamidoamine and the organic acid. In some examples, the amount of the organic acid in the composition or the cationic collector can be about 5 wt % to about 90 wt %, about 5 wt % to about 80 wt %, about 5 wt % to about 70 wt %, about 5 wt % to about 60 wt %, about 5 wt % to about 50 wt %, about 5 wt % to about 40 wt %, about 5 wt % to about 30 wt %, about 5 wt % to about 20 wt %, about 10 wt % to about 90 wt %, about 10 wt % to about 80 wt %, about 10 wt % to about 70 wt %, about 10 wt % to about 60 wt %, about 10 wt % to about 50 wt %, about 10 wt % to about 40 wt %, about 10 wt % to about 30 wt %, about 10 wt % to about 20 wt %, about 20 wt % to about 90 wt %, about 20 wt % to about 80 wt %, about 20 wt % to about 70 wt %, about 20 wt % to about 60 wt %, about 20 wt % to about 50 wt %, or about 20 wt % to about 40 wt %, based on the combined weight of the polyamidoamine and the organic acid.

In one specific example, the composition or the cationic collector can include about 10 wt % to about 60 wt % of the organic acid, e.g., acetic acid, and about 40 wt % to about 90 wt % of the polyamidoamine, based on the combined weight of the polyamidoamine and the organic acid. In another specific example, the composition or the cationic collector can include about 20 wt % to about 50 wt % of the organic acid and about 50 wt % to about 80 wt % of the polyamidoamine, based on the combined weight of the polyamidoamine and the organic acid. In another specific example, the composition or the cationic collector can include about 40 wt % to about 60 wt % of the organic acid and about 60 wt % to about 40 wt % of the polyamidoamine, based on the combined weight of the polyamidoamine and the organic acid. In another specific example, the composition or the cationic collector can include about 10 wt % to about 45 wt % of the organic acid and about 55 wt % to about 90 wt % of the polyamidoamine, based on the combined weight of the polyamidoamine and the organic acid.

In another example, the amount of the polyamidoamine in the composition or the cationic collector can be about 5 wt %, about 10 wt %, about 20 wt %, about 30 wt %, about 40 wt %, about 50 wt %, about 55 wt %, about 60 wt %, about 65 wt %, about 70 wt %, about 75 wt %, about 80 wt %, about 85 wt %, about 90 wt %, about 91 wt %, about 92 wt %, about 93 wt %, about 94 wt %, about 95 wt %, about 96 wt %, about 97 wt %, about 97.5 wt %, about 98 wt %, about 98.5 wt %, about 99 wt %, about 99.3 wt %, or about 99.5 wt %, based on the combined weight of the polyamidoamine, the organic acid, e.g., acetic acid, and the water. In some examples, the amount of the polyamidoamine in the composition or the cationic collector can be about 5 wt % to about 99 wt %, about 10 wt % to about 98 wt %, about 20 wt % to about 99 wt %, about 30 wt % to about 98 wt %, about 30 wt % to about 95 wt %, about 30 wt % to about 90 wt %, about 40 wt % to about 99 wt %, about 40 wt % to about 95 wt %, about 50 wt % to about 98 wt %, or about 50 wt % to about 95 wt %, based on the combined weight of the polyamidoamine, the organic acid, and the water. In one or more examples, the organic acid can be or include acetic acid in the cationic collector.

The amount of the organic acid, e.g., acetic acid, in the composition or the cationic collector can be about 1 wt %, about 2 wt %, about 3 wt %, about 4 wt %, about 5 wt %, about 6 wt %, about 7 wt %, about 8 wt %, about 9 wt %, about 10 wt %, about 12 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, or about 90 wt %, based on the combined weight of the polyamidoamine, the organic acid, and the water. In some examples, the amount of the organic acid in the composition or the cationic collector can be about 1 wt % to about 90 wt %, about 2 wt % to about 80 wt %, about 3 wt % to about 70 wt %, about 4 wt % to about 70 wt %, about 5 wt % to about 70 wt %, about 5 wt % to about 90 wt %, about 5 wt % to about 80 wt %, about 5 wt % to about 70 wt %, about 5 wt % to about 60 wt %, about 5 wt % to about 50 wt %, about 5 wt % to about 40 wt %, about 5 wt % to about 30 wt %, about 5 wt % to about 20 wt %, about 10 wt % to about 90 wt %, about 10 wt % to about 80 wt %, about 10 wt % to about 70 wt %, about 10 wt % to about 60 wt %, about 10 wt % to about 50 wt %, about 10 wt % to about 40 wt %, about 10 wt % to about 30 wt %, about 10 wt % to about 20 wt %, about 20 wt % to about 90 wt %, about 20 wt % to about 80 wt %, about 20 wt % to about 70 wt %, about 20 wt % to about 60 wt %, about 20 wt % to about 50 wt %, or about 20 wt % to about 40 wt %, based on the combined weight of the polyamidoamine, the organic acid, and the water.

The amount of the water in the composition or the cationic collector can be about 1 wt %, about 2 wt %, about 3 wt %, about 4 wt %, about 5 wt %, about 6 wt %, about 7 wt %, about 8 wt %, about 9 wt %, about 10 wt %, about 12 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, or about 90 wt %, based on the combined weight of the polyamidoamine, the organic acid, e.g., acetic acid, and the water. In some examples, the amount of the water in the composition or the cationic collector can be about 1 wt % to about 90 wt %, about 2 wt % to about 80 wt %, about 3 wt % to about 70 wt %, about 4 wt % to about 70 wt %, about 5 wt % to about 70 wt %, about 5 wt % to about 90 wt %, about 5 wt % to about 80 wt %, about 5 wt % to about 70 wt %, about 5 wt % to about 60 wt %, about 5 wt % to about 50 wt %, about 5 wt % to about 40 wt %, about 5 wt % to about 30 wt %, about 5 wt % to about 20 wt %, about 10 wt % to about 90 wt %, about 10 wt % to about 80 wt %, about 10 wt % to about 70 wt %, about 10 wt % to about 60 wt %, about 10 wt % to about 50 wt %, about 10 wt % to about 40 wt %, about 10 wt % to about 30 wt %, about 10 wt % to about 20 wt %, about 20 wt % to about 90 wt %, about 20 wt % to about 80 wt %, about 20 wt % to about 70 wt %, about 20 wt % to about 60 wt %, about 20 wt % to about 50 wt %, or about 20 wt % to about 40 wt %, based on the combined weight of the polyamidoamine, the organic acid, and the water.

In one specific example, the composition or the cationic collector can include about 2 wt % to about 50 wt % of the organic acid, e.g., acetic acid, about 2 wt % to about 50 wt % of water, and about 30 wt % to about 95 wt % of the polyamidoamine, based on the combined weight of the polyamidoamine, the organic acid, and the water. In another specific example, the composition or the cationic collector can include about 5 wt % to about 45 wt % of the organic acid, about 5 wt % to about 45 wt % of water, and about 40 wt % to about 90 wt % of the polyamidoamine, based on the combined weight of the polyamidoamine, the organic acid, and the water. In another specific example, the composition or the cationic collector can include about 10 wt % to about 40 wt % of the organic acid, about 10 wt % to about 40 wt % of water, and about 40 wt % to about 90 wt % of the polyamidoamine, based on the combined weight of the polyamidoamine, the organic acid, and the water. In another specific example, the composition or the cationic collector can include about 20 wt % to about 60 wt % of the organic acid, about 20 wt % to about 60 wt % of water, and about 30 wt % to about 80 wt % of the polyamidoamine, based on the combined weight of the polyamidoamine, the organic acid, and the water.

In one or more examples, the composition or the cationic collector can include one or more polyamidoamines which can be or include one or more polyalkylene polyamidoamines. Illustrative polyalkylene polyamidoamines can include, but are not limited to, polyethylene polyamidoamines, polypropylene polyamidoamines, polybutylene polyamidoamines, or any combination thereof. In some examples, the composition or the cationic collector can include one or more polyamidoamines which can be or include one or more polyethylene polyamidoamines. Illustrative polyethylene polyamidoamines can include, but are not limited to, polyethylene diamidoamines, polyethylene triamidoamines, polyethylene polyamidoamines with four or more amido groups, or any mixture thereof. In some examples, the composition or the cationic collector can include one or more polyamidoamines which can be or include one or more mixtures of polyethylene diamidoamines and polyethylene triamidoamines.

The mixture of polyethylene diamidoamines and polyethylene triamidoamines can include about 0.5 mol %, about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, about 20 mol %, about 25 mol %, about 30 mol %, about 35 mol %, about 40 mol %, about 45 mol %, about 50 mol %, about 55 mol %, about 60 mol %, about 65 mol %, about 70 mol %, about 75 mol %, about 80 mol %, about 81 mol %, about 82 mol %, about 83 mol %, about 84 mol %, about 85 mol %, about 86 mol %, about 87 mol %, about 88 mol %, about 89 mol %, about 91 mol %, about 92 mol %, about 93 mol %, about 94 mol %, about 95 mol %, about 96 mol %, about 97 mol %, about 98 mol %, about 99 mol %, about 99.1 mol %, about 99.2 mol %, about 99.3 mol %, about 99.4 mol %, about 99.5 mol %, about 99.6 mol %, about 99.7 mol %, about 99.8 mol %, or about 99.9 mol % of the polyethylene diamidoamines, based on the combined moles of the polyethylene diamidoamines and the polyethylene triamidoamines. For example, the mixture of polyethylene diamidoamines and polyethylene triamidoamines can include about 5 mol % to about 99.5 mol %, about 10 mol % to about 99 mol %, about 20 mol % to about 95 mol %, about 30 mol % to about 95 mol %, about 40 mol % to about 95 mol %, about 50 mol % to about 95 mol %, about 60 mol % to about 95 mol %, about 70 mol % to about 95 mol %, about 80 mol % to about 95 mol %, about 90 mol % to about 95 mol %, about 60 mol % to about 90 mol %, about 70 mol % to about 90 mol %, about 80 mol % to about 90 mol %, about 85 mol % to about 99.5 mol %, about 86 mol % to about 99.5 mol %, about 87 mol % to about 99.5 mol %, about 88 mol % to about 99.5 mol %, about 89 mol % to about 99.5 mol %, about 90 mol % to about 99.5 mol %, about 91 mol % to about 99.5 mol %, about 92 mol % to about 99.5 mol %, about 93 mol % to about 99.5 mol %, about 94 mol % to about 99.5 mol %, about 95 mol % to about 99.5 mol %, about 96 mol % to about 99.5 mol %, about 97 mol % to about 99.5 mol %, about 98 mol % to about 99.5 mol %, or about 99 mol % to about 99.5 mol % of the polyethylene diamidoamines, based on the combined moles of the polyethylene diamidoamines and the polyethylene triamidoamines.

The mixture of polyethylene diamidoamines and polyethylene triamidoamines can include about 0.1 mol %, about 0.2 mol %, about 0.3 mol %, about 0.4 mol %, about 0.5 mol %, about 0.6 mol %, about 0.7 mol %, about 0.8 mol %, about 0.9 mol %, about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, about 20 mol %, about 25 mol %, about 30 mol %, about 35 mol %, about 40 mol %, about 45 mol %, about 50 mol %, about 55 mol %, about 60 mol %, about 65 mol %, about 70 mol %, about 75 mol %, about 80 mol %, about 85 mol %, about 90 mol %, or about 95 mol % of the polyethylene triamidoamines, based on the combined moles of the polyethylene diamidoamines and the polyethylene triamidoamines. For example, the mixture of polyethylene diamidoamines and polyethylene triamidoamines can include about 0.5 mol % to about 95 mol %, about 1 mol % to about 90 mol %, about 1 mol % to about 80 mol %, about 1 mol % to about 70 mol %, about 1 mol % to about 60 mol %, about 1 mol % to about 50 mol %, about 1 mol % to about 40 mol %, about 1 mol % to about 30 mol %, about 1 mol % to about 20 mol %, about 1 mol % to about 10 mol %, about 5 mol % to about 90 mol %, about 5 mol % to about 80 mol %, about 5 mol % to about 70 mol %, about 5 mol % to about 60 mol %, about 5 mol % to about 50 mol %, about 5 mol % to about 40 mol %, about 5 mol % to about 30 mol %, about 5 mol % to about 20 mol %, about 5 mol % to about 10 mol %, about 0.5 mol % to about 30 mol %, about 0.5 mol % to about 25 mol %, about 0.5 mol % to about 20 mol %, about 0.5 mol % to about 19 mol %, about 0.5 mol % to about 18 mol %, about 0.5 mol % to about 17 mol %, about 0.5 mol % to about 16 mol %, about 0.5 mol % to about 15 mol %, about 0.5 mol % to about 14 mol %, about 0.5 mol % to about 13 mol %, about 0.5 mol % to about 12 mol %, about 0.5 mol % to about 11 mol %, about 0.5 mol % to about 10 mol %, about 0.5 mol % to about 9 mol %, about 0.5 mol % to about 8 mol %, about 0.5 mol % to about 7 mol %, about 0.5 mol % to about 6 mol %, about 0.5 mol % to about 5 mol %, about 0.5 mol % to about 4 mol %, about 0.5 mol % to about 3 mol %, about 0.5 mol % to about 2 mol %, or about 0.5 mol % to about 1 mol % of the polyethylene triamidoamines, based on the combined moles of the polyethylene diamidoamines and the polyethylene triamidoamines.

In some examples, the mixtures of polyethylene diamidoamines and polyethylene triamidoamines can include about 70 mol % to about 99.5 mol % of the polyethylene triamidoamines and about 0.5 mol % to about 30 mol % of the polyethylene triamidoamines, based on the combined weight of the polyethylene diamidoamines and the polyethylene triamidoamines. In other examples, the mixtures of polyethylene diamidoamines and polyethylene triamidoamines can include about 80 mol % to about 99.5 mol % of the polyethylene triamidoamines and about 0.5 mol % to about 20 mol % of the polyethylene triamidoamines, based on the combined weight of the polyethylene diamidoamines and the polyethylene triamidoamines. In other examples, the mixtures of polyethylene diamidoamines and polyethylene triamidoamines can include about 90 mol % to about 99.5 mol % of the polyethylene triamidoamines and about 0.5 mol % to about 10 mol % of the polyethylene triamidoamines, based on the combined weight of the polyethylene diamidoamines and the polyethylene triamidoamines.

In one or more examples, the composition or the cationic collector can include one, two, three, or more polyamidoaminates and have a free flowing viscosity. The composition or the cationic collector can have a viscosity of about 10 cP, about 20 cP, about 30 cP, about 40 cP, about 50 cP, about 60 cP, about 70 cP, about 80 cP, about 90 cP, about 100 cP, about 110 cP, about 120 cP, about 130 cP, about 140 cP, about 150 cP, about 160 cP, about 170 cP, about 180 cP, about 190 cP, about 200 cP, about 210 cP, about 220 cP, about 230 cP, about 240 cP, about 250 cP, about 260 cP, about 270 cP, about 280 cP, about 290 cP, about 300 cP to about 350 cP, about 400 cP, about 450 cP, about 500 cP, about 600 cP, about 700 cP, or about 800 cP at a temperature of about 25° C. For example, the composition or the cationic collector can have a viscosity of about 10 cP to about 500 cP, about 10 cP to about 450 cP, about 10 cP to about 400 cP, about 10 cP to about 350 cP, about 10 cP to about 300 cP, about 10 cP to about 250 cP, about 10 cP to about 200 cP, about 10 cP to about 150 cP, about 10 cP to about 125 cP, about 10 cP to about 100 cP, about 10 cP to about 80 cP, about 30 cP to about 500 cP, about 30 cP to about 450 cP, about 30 cP to about 400 cP, about 30 cP to about 350 cP, about 30 cP to about 300 cP, about 30 cP to about 250 cP, about 30 cP to about 200 cP, about 30 cP to about 150 cP, about 30 cP to about 125 cP, about 30 cP to about 100 cP, about 30 cP to about 80 cP, about 50 cP to about 500 cP, about 50 cP to about 450 cP, about 50 cP to about 400 cP, about 50 cP to about 350 cP, about 50 cP to about 300 cP, about 50 cP to about 250 cP, about 50 cP to about 200 cP, about 50 cP to about 150 cP, about 50 cP to about 125 cP, about 50 cP to about 100 cP, or about 50 cP to about 80 cP at a temperature of about 25° C. In some examples, the composition or the cationic collector can have a viscosity of about 10 cP to less than 500 cP, about 10 cP to less than 400 cP, about 10 cP to less than 300 cP, about 10 cP to less than 250 cP, about 10 cP to less than 200 cP, about 10 cP to less than 150 cP, about 10 cP to less than 125 cP, about 10 cP to less than 100 cP, about 10 cP to less than 80 cP, about 50 cP to less than 500 cP, about 50 cP to less than 400 cP, about 50 cP to less than 300 cP, about 50 cP to less than 250 cP, about 50 cP to less than 200 cP, about 50 cP to less than 150 cP, about 50 cP to less than 125 cP, about 50 cP to less than 100 cP, or about 50 cP to about 80 cP at a temperature of about 25° C. In other examples, the composition or the cationic collector can have a viscosity of about 400 cP to about 5,000 cP, about 400 cP to about 4,500 cP, about 400 cP to about 4,000 cP, about 400 cP to about 3,500 cP, about 400 cP to about 3,000 cP, about 400 cP to about 2,500 cP, about 400 cP to about 2,000 cP, about 400 cP to about 1,500 cP, about 400 cP to about 1,250 cP, about 400 cP to about 1,000 cP, about 400 cP to about 800 cP, about 500 cP to about 5,000 cP, about 500 cP to about 4,500 cP, about 500 cP to about 4,000 cP, about 500 cP to about 3,500 cP, about 500 cP to about 3,000 cP, about 500 cP to about 2,500 cP, about 500 cP to about 2,000 cP, about 500 cP to about 1,500 cP, about 500 cP to about 1,250 cP, about 500 cP to about 1,000 cP, about 500 cP to about 800 cP, about 700 cP to about 5,000 cP, about 700 cP to about 4,500 cP, about 700 cP to about 4,000 cP, about 700 cP to about 3,500 cP, about 700 cP to about 3,000 cP, about 700 cP to about 2,500 cP, about 700 cP to about 2,000 cP, about 700 cP to about 1,500 cP, about 700 cP to about 1,250 cP, about 700 cP to about 1,000 cP, or about 700 cP to about 800 cP at a temperature of about 25° C.

The composition or the cationic collector can have a viscosity of about 10 cP, about 20 cP, or about 30 cP, to about 40 cP, about 50 cP, about 60 cP, about 70 cP, about 80 cP, about 90 cP, about 100 cP, about 110 cP, about 120 cP, about 130 cP, about 140 cP, about 150 cP, about 160 cP, about 170 cP, about 180 cP, about 190 cP, about 200 cP, about 210 cP, about 220 cP, about 230 cP, about 240 cP, about 250 cP, about 260 cP, about 270 cP, about 280 cP, about 290 cP, or about 300 cP at a temperature of about 80° C. For example, the composition or the cationic collector can have a viscosity of about 10 cP to about 300 cP, about 10 cP to about 250 cP, about 10 cP to about 200 cP, about 10 cP to about 150 cP, about 10 cP to about 125 cP, about 10 cP to about 100 cP, about 10 cP to about 80 cP, about 10 cP to about 60 cP, about 10 cP to about 50 cP, about 20 cP to about 300 cP, about 20 cP to about 250 cP, about 20 cP to about 200 cP, about 20 cP to about 150 cP, about 20 cP to about 125 cP, about 20 cP to about 100 cP, about 20 cP to about 80 cP, about 20 cP to about 60 cP, about 20 cP to about 50 cP, about 30 cP to about 300 cP, about 30 cP to about 250 cP, about 30 cP to about 200 cP, about 30 cP to about 150 cP, about 30 cP to about 125 cP, about 30 cP to about 100 cP, about 30 cP to about 80 cP, about 30 cP to about 60 cP, or about 30 cP to about 50 cP at a temperature of about 80° C. In some examples, the composition or the cationic collector can have a viscosity of about 10 cP to less than 300 cP, about 10 cP to less than 250 cP, about 10 cP to less than 200 cP, about 10 cP to less than 150 cP, about 10 cP to less than 125 cP, about 10 cP to less than 100 cP, about 10 cP to less than 80 cP, about 10 cP to less than 60 cP, or about 10 cP to less than 50 cP at a temperature of about 80° C.

In other examples, when the composition or the cationic collector includes the organic acid the cationic collector can have a viscosity of about 10 cP, about 20 cP, about 30 cP, about 40 cP, about 50 cP, about 60 cP, about 70 cP, about 80 cP, about 90 cP, about 100 cP, about 110 cP, about 120 cP, about 130 cP, about 140 cP, about 150 cP, about 160 cP, about 170 cP, about 180 cP, about 190 cP, about 200 cP, about 210 cP, about 220 cP, about 230 cP, about 240 cP, about 250 cP, about 260 cP, about 270 cP, about 280 cP, about 290 cP, about 300 cP to about 350 cP, about 400 cP, about 450 cP, about 500 cP, about 600 cP, about 700 cP, or about 800 cP at a temperature of about 25° C. when the cationic collector includes about 2 wt % to about 50 wt % of the organic acid, e.g., acetic acid, about 2 wt % to about 50 wt % of water, and about 30 wt % to about 95 wt % of the polyamidoamine, based on the combined weight of the polyamidoamine, the organic acid, and the water. In other examples, the when the composition or the cationic collector includes the organic acid the composition or the cationic collector can have a viscosity of about 10 cP, about 20 cP, about 30 cP, about 40 cP, about 50 cP, about 60 cP, about 70 cP, about 80 cP, about 90 cP, about 100 cP, about 110 cP, about 120 cP, about 130 cP, about 140 cP, about 150 cP, about 160 cP, about 170 cP, about 180 cP, about 190 cP, about 200 cP, about 210 cP, about 220 cP, about 230 cP, about 240 cP, about 250 cP, about 260 cP, about 270 cP, about 280 cP, about 290 cP, about 300 cP to about 350 cP, about 400 cP, about 450 cP, about 500 cP, about 600 cP, about 700 cP, or about 800 cP at a temperature of about 25° C. when the composition or the cationic collector includes about 20 wt % to about 60 wt % of the organic acid, about 20 wt % to about 60 wt % of water, and about 30 wt % to about 80 wt % of the polyamidoamine, based on the combined weight of the polyamidoamine, the organic acid, and the water.

The viscosity of the various compositions discussed and described herein can be determined using a viscometer at a specified temperature, such as about 25° C. or about 80° C. For example, a viscometer, Model DV-II+, commercially available from the Brookfield Company, with a small sample adapter with, for example, a number 3 spindle, can be used. The small sample adapter can allow the sample to be cooled or heated by the chamber jacket to maintain the temperature of the sample surrounding the spindle at a temperature of about 25° C. (unless otherwise noted).

In one or more examples, the amount of the composition or the cationic collector in the aqueous mixture can be about 0.0005 wt %, about 0.001 wt %, about 0.005 wt %, about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.11 wt %, about 0.12 wt %, about 0.13 wt %, about 0.14 wt %, about 0.15 wt %, about 0.16 wt %, about 0.17 wt %, about 0.18 wt %, about 0.19 wt %, about 0.2 wt %, about 0.25 wt %, about 0.3 wt %, about 0.35 wt %, about 0.4 wt %, about 0.5 wt %, about 0.6 wt %, about 0.7 wt %, about 0.8 wt %, about 0.9 wt %, about 1 wt %, about 1.5 wt %, about 2 wt %, about 2.5 wt %, about 3 wt %, about 3.5 wt %, about 4 wt %, about 4.5 wt %, about 5 wt %, about 6 wt %, about 7 wt %, about 8 wt %, about 9 wt %, or about 10 wt %, based on the weight of the ore, e.g., a phosphorous ore. In some examples, the amount of the composition or the cationic collector in the aqueous mixture can be about 0.001 wt % to about 10 wt %, about 0.005 wt % to about 5 wt %, about 0.005 wt % to about 2 wt %, about 0.005 wt % to about 1 wt %, about 0.005 wt % to about 0.5 wt %, about 0.005 wt % to about 0.1 wt %, about 0.005 wt % to about 0.09 wt %, or about 0.005 wt % to about 0.05 wt %%, based on the weight of the ore. In other examples, the amount of the composition or the cationic collector in the aqueous mixture can be greater than 0.001 wt % to about 10 wt %, greater than 0.005 wt % to about 5 wt %, greater than 0.005 wt % to about 2 wt %, greater than 0.005 wt % to about 1 wt %, greater than 0.005 wt % to about 0.5 wt %, greater than 0.005 wt % to about 0.1 wt %, greater than 0.005 wt % to about 0.09 wt %, or greater than 0.005 wt % to about 0.05 wt %%, based on the weight of the ore. In other examples, the amount of the composition or the cationic collector in the aqueous mixture can be about 0.001 wt % to less than 10 wt %, about 0.005 wt % to less than 5 wt %, about 0.005 wt % to less than 2 wt %, about 0.005 wt % to less than 1 wt %, about 0.005 wt % to less than 0.5 wt %, about 0.005 wt % to less than 0.1 wt %, about 0.005 wt % to less than 0.09 wt %, or about 0.005 wt % to less than 0.05 wt %%, based on the weight of the ore.

In one or more examples, the amount of the polyamidoamine in the aqueous mixture can be about 0.0001 wt %, about 0.0005 wt %, about 0.001 wt %, about 0.005 wt %, about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.11 wt %, about 0.12 wt %, about 0.13 wt %, about 0.14 wt %, about 0.15 wt %, about 0.16 wt %, about 0.17 wt %, about 0.18 wt %, about 0.19 wt %, about 0.2 wt %, about 0.25 wt %, about 0.3 wt %, about 0.35 wt %, about 0.4 wt %, about 0.5 wt %, about 0.6 wt %, about 0.7 wt %, about 0.8 wt %, about 0.9 wt %, about 1 wt %, about 1.5 wt %, about 2 wt %, about 2.5 wt %, about 3 wt %, about 3.5 wt %, about 4 wt %, about 4.5 wt %, about 5 wt %%, based on the weight of the ore, e.g., a phosphorous ore. In some examples, the amount of the polyamidoamine in the aqueous mixture can be about 0.0001 wt % to about 2 wt %, about 0.0005 wt % to about 1 wt %, about 0.001 wt % to about 1 wt %, about 0.005 wt % to about 1 wt %, about 0.005 wt % to about 0.5 wt %, about 0.005 wt % to about 0.1 wt %, about 0.005 wt % to about 0.09 wt %, or about 0.005 wt % to about 0.05 wt %%, based on the weight of the ore. In other examples, the amount of the polyamidoamine in the aqueous mixture can be greater than 0.0001 wt % to about 2 wt %, greater than 0.0005 wt % to about 1 wt %, greater than 0.001 wt % to about 1 wt %, greater than 0.005 wt % to about 1 wt %, greater than 0.005 wt % to about 0.5 wt %, greater than 0.005 wt % to about 0.1 wt %, greater than 0.005 wt % to about 0.09 wt %, or greater than 0.005 wt % to about 0.05 wt %%, based on the weight of the ore. In other examples, the amount of the polyamidoamine in the aqueous mixture can be about 0.0001 wt % to less than 2 wt %, about 0.0005 wt % to less than 1 wt %, about 0.001 wt % to less than 1 wt %, about 0.005 wt % to less than 1 wt %, about 0.005 wt % to less than 0.5 wt %, about 0.005 wt % to less than 0.1 wt %, about 0.005 wt % to less than 0.09 wt %, or about 0.005 wt % to less than 0.05 wt %%, based on the weight of the ore.

In one or more examples, the amount of the organic acid, e.g., acetic acid, in the aqueous mixture can be about 0.0001 wt %, about 0.0005 wt %, about 0.001 wt %, about 0.005 wt %, about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.11 wt %, about 0.12 wt %, about 0.13 wt %, about 0.14 wt %, about 0.15 wt %, about 0.16 wt %, about 0.17 wt %, about 0.18 wt %, about 0.19 wt %, about 0.2 wt %, about 0.25 wt %, about 0.3 wt %, about 0.35 wt %, about 0.4 wt %, about 0.5 wt %, about 0.6 wt %, about 0.7 wt %, about 0.8 wt %, about 0.9 wt %, about 1 wt %, about 1.5 wt %, about 2 wt %, about 2.5 wt %, about 3 wt %, about 3.5 wt %, about 4 wt %, about 4.5 wt %, about 5 wt %%, based on the weight of the ore, e.g., a phosphorous ore. In some examples, the amount of the organic acid in the aqueous mixture can be about 0.0001 wt % to about 2 wt %, about 0.0005 wt % to about 1 wt %, about 0.001 wt % to about 1 wt %, about 0.005 wt % to about 1 wt %, about 0.005 wt % to about 0.5 wt %, about 0.005 wt % to about 0.1 wt %, about 0.005 wt % to about 0.09 wt %, or about 0.005 wt % to about 0.05 wt %%, based on the weight of the ore. In some examples, the amount of the organic acid in the aqueous mixture can be greater than 0.0001 wt % to about 2 wt %, greater than 0.0005 wt % to about 1 wt %, greater than 0.001 wt % to about 1 wt %, greater than 0.005 wt % to about 1 wt %, greater than 0.005 wt % to about 0.5 wt %, greater than 0.005 wt % to about 0.1 wt %, greater than 0.005 wt % to about 0.09 wt %, or greater than 0.005 wt % to about 0.05 wt %%, based on the weight of the ore. In some examples, the amount of the organic acid in the aqueous mixture can be about 0.0001 wt % to less than 2 wt %, about 0.0005 wt % to less than 1 wt %, about 0.001 wt % to less than 1 wt %, about 0.005 wt % to less than 1 wt %, about 0.005 wt % to less than 0.5 wt %, about 0.005 wt % to less than 0.1 wt %, about 0.005 wt % to less than 0.09 wt %, or about 0.005 wt % to less than 0.05 wt %%, based on the weight of the ore. In one or more examples, the organic acid can be or include acetic acid, e.g., glacial acetic acid.

The aqueous mixtures which can include water, one or more ores such as a phosphorous ore, one or more polyamidoamines, and one or more organic acids, e.g., acetic acid, including aqueous suspensions, dispersions, slurries, solutions, or mixtures, can be conditioned for a given time period during and between steps of combining components. Conditioning the aqueous mixture upon the addition of water, one or more ores, one or more polyamidoamines, and one or more organic acids, e.g., acetic acid, can facilitate contact between the components. Conditioning can include, but is not limited to, agitating the aqueous mixture for a given time period prior to subjecting the aqueous mixture to separation or collection techniques. For example, the aqueous mixtures can be stirred, blended, mixed, air or gas bubbled, or otherwise agitated for a time of about 30 seconds, about 1 minute, about 2 minutes, about 3 minutes, about 4 minutes, about 5 minutes, about 6 minutes, about 7 minutes, about 8 minutes, about 9 minutes, about 10 minutes, about 12 minutes, about 15 minutes, about 20 minutes, about 30 minutes, about 1 hour, or about 24 hours. Conditioning the aqueous mixture can also include heating (or cooling) mixture to a temperature of about 15° C., about 20° C., about 25° C., about 30° C., about 35° C., about 60° C., about 80° C., or about 95° C.

Conditioning the aqueous mixture can also include adjusting the pH values of any of portions of and including the aqueous mixtures. The aqueous mixture containing the ore, e.g., phosphorous ore, the polyamidoamine, the organic acid, e.g., acetic acid, and water can be maintained at or adjusted to have a pH value of greater than 7, such as about 7.5, about 8, about 8.5, about 9, about 9.5, about 10, about 10.5, about 11, about 11.5, about 12, about 12.5, or about 13. In one or more examples, the pH value of the aqueous mixture can be or can be adjusted to about 8.5 to about 10.5, about 9 to about 10, about 9.2 to about 9.8, or about 9.5. In other examples, the pH value of the aqueous mixture can be or can be adjusted to about 8.5 to about 10.5, about 9 to about 10, about 9.2 to about 9.8, or about 9.5. Any one or combination of acid and/or base compounds can be combined with the mixtures to adjust the pH values thereof.

Illustrative acid compounds that can be used to maintain or adjust the pH value of any of the aqueous mixtures can include, but are not limited to, one or more mineral acids, one or more organic acids, one or more acid salts, or any mixture thereof. Illustrative mineral acids can include, but are not limited to, hydrochloric acid, nitric acid, phosphoric acid, sulfuric acid, or any mixture thereof. Illustrative organic acids can include, but are not limited to, acetic acid, formic acid, citric acid, oxalic acid, uric acid, lactic acid, or any mixture thereof. Illustrative acid salts can include, but are not limited to, ammonium sulfate, sodium bisulfate, sodium metabisulfite, or any mixture thereof.

Illustrative base compounds that can be used to maintain or adjust the pH value of any of the aqueous mixtures can include, but are not limited to, hydroxides, carbonates, ammonia, amines, or any mixture thereof. Illustrative hydroxides can include, but are not limited to, sodium hydroxide, potassium hydroxide, ammonium hydroxide, e.g., aqueous ammonia, lithium hydroxide, and cesium hydroxide. Illustrative carbonates can include, but are not limited to, sodium carbonate, sodium bicarbonate, potassium carbonate, and ammonium carbonate. Illustrative amines can include, but are not limited to, trimethylamine, triethylamine, triethanolamine, diisopropylethylamine (Hunig's base), pyridine, 4-dimethylaminopyridine (DMAP), and 1,4-diazabicyclo[2.2.2]octane (DABCO).

In one or more examples, the aqueous mixture or slurry can be aerated in a conventional flotation machine or bank of rougher cells to float phosphates or other phosphorous containing materials. Any conventional flotation unit can be employed. The composition or the cationic collector can be used to separate a wide variety of contaminants from a liquid, e.g., water. For example, the composition or the cationic collector can be used to separate siliceous contaminants such as sand, clay, and/or ash from aqueous liquid suspensions or slurries containing one or more of these siliceous contaminants. Aqueous suspensions or slurries can therefore be treated with the cationic collector allowing for the effective separation of at least a portion of the contaminants, in a contaminant-rich fraction, to provide a purified liquid. The contaminant-rich fraction contains a higher percentage of solid contaminants than originally present in the aqueous mixture or slurry. Conversely, the purified liquid has a lower percentage of solid contaminants than originally present in the aqueous mixture or slurry.

The treatment can involve adding an effective amount of the composition or the cationic collector to interact with and either coagulate or flocculate one or more solid contaminants into larger agglomerates. An effective amount can be readily determined depending, at least in part, on a number of variables, e.g., the type and concentration of contaminant. In other examples, the treatment can involve contacting the aqueous mixture or slurry continuously with a fixed bed of the composition or the cationic collector, in solid form.

During or after the treatment of the aqueous mixture or slurry with the composition or the cationic collector, the coagulated or flocculated solid contaminant (which can now be, for example, in the form of larger, agglomerated particles or flocs) can be removed. Removal can be effected by flotation (with or without the use of rising air bubbles, such as in a froth flotation. Filtration or straining can also be an effective means for removing the agglomerated flocs of solid particulates on the surface of the aqueous mixture or slurry.

Considering froth flotation in more detail, froth flotation is a separation process based on differences in the tendency of various materials to associate with rising air bubbles. The composition or cationic collector and optionally a dispersant, a depressant, and/or other additives can be combined with water and an ore that includes one or more contaminants to produce an aqueous slurry or other mixture. A gas, e.g., air, can be flowed, forced, or otherwise passed through the mixture. Some materials (e.g., value minerals) will, relative to others (e.g., contaminants), exhibit preferential affinity for air bubbles, causing them to rise to the surface of the aqueous slurry, where they can be collected in a froth concentrate. A degree of separation is thereby provided. In “reverse” froth flotation, it is the contaminant that can preferentially float and concentrated at the surface, with the ore and/or other value material concentrated in the bottoms. The relatively hydrophobic fraction of the material can have a selective affinity for the rising bubbles and can float to the surface, where it can be skimmed off and recovered. The relatively hydrophilic fraction of the material can flow or otherwise move toward the bottom of the aqueous mixture and can be recovered as a bottoms fraction. Froth flotation is a separation process well known to those skilled in the art.

As used herein, the term “purifying” broadly refers to any process for beneficiation, upgrading, and/or recovering, a value material as described herein, such as phosphates or other phosphorous containing materials. In some examples, the aqueous mixture or slurry can include the clay-containing aqueous suspensions or brines, which accompany ore refinement processes, including those described above. The production of purified phosphate from mined calcium phosphate rock, for example, generally relies on multiple separations of solid particulates from aqueous media, whereby such separations can be improved using the cationic collector. In the overall process, calcium phosphate can be mined from deposits and the phosphate rock can be initially recovered in a matrix containing sand and clay impurities. The matrix can be mixed with water to form a slurry, which after mechanical agitation, can be screened to retain phosphate pebbles and to allow fine clay particles to pass through as a clay slurry effluent with large amounts of water.

These clay-containing effluents can have high flow rates and generally carry less than 10 wt % of solids, e.g., about 1 wt % to about 5 wt % of solids. The dewatering, e.g., by settling or filtration, of this waste clay, which allows for recycle of the water, poses a significant challenge for reclamation. The time required to dewater the clay, however, can be decreased through treatment of the clay slurry effluent, obtained in the production of phosphate, with the cationic collector. Reduction in the clay settling time allows for efficient re-use of the purified water, obtained from clay dewatering, in the phosphate production operation. In one example of the purification method, where the aqueous mixture or slurry is a clay-containing effluent slurry from a phosphate production facility, the purified liquid can contain less than 1 wt % solids after a settling or dewatering time of less than 1 month.

In addition to the phosphate pebbles and clay slurry effluent that can be produced by screening the slurry of the matrix that can contain sand and clay impurities described above, a mixture of sand and finer particles of phosphate can also obtained in the initial processing of mined phosphate matrix. The sand and phosphate can be separated by froth flotation which, as described above, can be improved using the cationic collector as a depressant for the sand.

In one or more examples, the phosphate material that can be collected, recovered or otherwise purified from the aqueous mixture due to the cationic collector can be compared to the initial or total amount of the phosphate material contained in the phosphorous ore. For example, the collected or recovered phosphate material can be about 90 wt %, about 91 wt %, about 92 wt %, about 93 wt %, about 94 wt %, about 95 wt %, about 96 wt %, about 97 wt %, about 97.1 wt %, about 97.2 wt %, about 97.3 wt %, about 97.4 wt %, about 97.5 wt %, about 97.6 wt %, about 97.7 wt %, about 97.8 wt %, or about 97.9 wt %, about 98 wt %, about 98.1 wt %, about 98.2 wt %, about 98.3 wt %, about 98.4 wt %, about 98.5 wt %, about 98.6 wt %, about 98.7 wt %, about 98.8 wt %, about 98.9 wt %, about 99 wt %, about 99.1 wt %, about 99.2 wt %, about 99.3 wt %, about 99.4 wt %, about 99.5 wt %, about 99.6 wt %, about 99.7 wt %, about 99.8 wt %, or about 99.9 wt % of the total phosphate material contained in the phosphorous ore. In other examples, the collected or recovered phosphate material can be about 90 wt % to about 99.9 wt %, about 91 wt % to about 99.9 wt %, about 92 wt % to about 99.9 wt %, about 93 wt % to about 99.9 wt %, about 94 wt % to about 99.9 wt %, about 95 wt % to about 99.9 wt %, about 96 wt % to about 99.9 wt %, about 97 wt % to about 99.9 wt %, about 98 wt % to about 99.9 wt %, about 99 wt % to about 99.9 wt %, about 99.1 wt % to about 99.9 wt %, about 99.2 wt % to about 99.9 wt %, about 99.3 wt % to about 99.9 wt %, about 99.4 wt % to about 99.9 wt %, about 99.5 wt % to about 99.9 wt %, about 99.6 wt % to about 99.9 wt %, about 99.7 wt % to about 99.9 wt %, about 95 wt % to about 99.7 wt %, about 96 wt % to about 99.7 wt %, about 97 wt % to about 99.7 wt %, about 98 wt % to about 99.7 wt %, about 99 wt % to about 99.7 wt %, about 95 wt % to about 99.5 wt %, about 96 wt % to about 99.5 wt %, about 97 wt % to about 99.5 wt %, about 98 wt % to about 99.5 wt %, or about 99 wt % to about 99.5 wt % of the total phosphate material contained in the phosphorous ore. In one specific example, the collected phosphate material can be about 98 wt % to about 99.9 wt % of the total phosphate material contained in the phosphorous ore.

In some examples, a tail material can be submerged, flocculated, sunk, suspended, or otherwise rejected or not floated at the top of the aqueous mixture or slurry. The tail material can include acid insoluble materials and/or other impurities formerly contained in the phosphorous or phosphate containing ores, rocks, minerals, or other materials. The tail material flocculated in the aqueous mixture can be collected or otherwise recovered, separately from the recovered phosphate material. The tail material can generally be less than 99 wt % of the total acid insolubles (AI) contained in the phosphorous ore. For example, the tail material can be less than 97 wt %, less than 95 wt %, less than 90 wt %, less than 85 wt %, less than 80 wt %, less than 75 wt %, less than 70 wt %, less than 65 wt %, less than 60 wt %, less than 65 wt %, less than 50 wt % to about 40 wt %, about 30 wt %, about 20 wt %, about 10 wt %, about 5 wt %, or less, based on the total acid insolubles contained in the phosphorous ore. In some examples, the acid insolubles can be about 10 wt % to less than 97 wt %, about 25 wt % to less than 95 wt %, about 40 wt % to less than 95 wt %, about 50 wt % to less than 95 wt %, about 60 wt % to less than 95 wt %, about 70 wt % to less than 95 wt %, about 80 wt % to less than 95 wt %, about 90 wt % to less than 95 wt %, about 50 wt % to about 90 wt %, about 60 wt % to about 90 wt %, about 70 wt % to about 90 wt %, or about 80 wt % to about 90 wt %, based on the total acid insolubles contained in the phosphorous ore. In one specific example, a tail material can be collected or recovered that can be flocculated on the bottom of the aqueous mixture, where the tail material can include acid insolubles, and the acid insolubles can be about 70 wt % to about 90 wt % of the total acid insolubles contained in the phosphorous ore.

The separation efficiency is defined as Eₛ=R_{PO}−R_{AI}, where R_{PO} is the ratio of the total weight of the recovered phosphate material over the total weight of the phosphate material contained in the phosphorous ore and R_{AI} is the ratio of the total weight of the recovered acid insolubles over the total weight of the phosphorous ore. The cationic collector can provide a separation efficiency for purified materials, including phosphate, of about 50 wt % of greater, such as about 55 wt %, about 60 wt %, about 65 wt %, about 70 wt %, about 75 wt %, about 80 wt %, about 81 wt %, about 82 wt %, about 83 wt %, about 84 wt %, about 85 wt %, about 86 wt %, about 87 wt %, about 88 wt %, about 89 wt %, about 90 wt %, about 91 wt %, about 92 wt %, about 93 wt %, about 94 wt %, about 95 wt %, about 96 wt %, about 97 wt %, about 98 wt %, or about 99 wt %.

In one or more examples, the compositions or the cationic collectors having one or more polyamidoamines which incorporate at least one naphthenate group can be used to increase the flotation of silicate materials, such as sand. The polyamidoamine can be formed by reacting one or more polyamines with naphthenic acid and optionally one or more other fatty acids or other carboxylic acids. For example, the polyamidoamine can be formed by reacting DETA, TETA, TEPA, and/or PEHA with naphthenic acid and one or more of lauric acid, isostearic acid, oleic acid, linoleic acid, TOFA, and/or other fatty acids.

In one example, the polyamidoamines having the chemical formula (D) where R² is a naphthenate group, can be or include one or more amidoamines having the chemical formula (0):

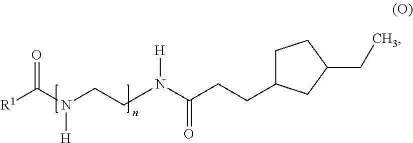

where R¹ and n are defined as above for the chemical formula (D). Naphthenic acid can generally include mixtures of carboxylic acid compounds having cyclopentyl, cyclohexyl cyclic, and/or other cyclic motifs with C6 to C24 chains, e.g., backbone chains or carboxylic acid chains, such as C9 to C20 chains, C9 to C19 chains, and/or C10 to C16 chains. Naphthenic acids and naphthenate groups can include one or more cyclopentyl carboxylic acids or one or more cyclohexyl carboxylic acids that have one or more C9 to C20 chains, C9 to C19 chains, and/or C10 to C16 chains as backbone chains or carboxylic acid chains.

In some specific examples, the polyamidoamines can have the chemical formula (O), where R¹ can be a C6 to C24 chain or a C8 to C24 chain and n can be 2, 3, 4, or 5. In other examples, the polyamidoamines can have the chemical formula (O), where R¹ can be a C10 to C24 chain having 0 to 2 unsaturated bonds and n can be 2, 3, or 4. In other examples, the polyamidoamines can have the chemical formula (O), where R¹ can be C₉H₁₉, C₉H₁₇, C₉H₁₅, C₉H₁₃, C₁₁H₂₃, C₁₁H₂₁, C₁₅H₃₃, C₁₅H₃₁, C₁₅H₂₉, C₁₇H₃₅, C₁₇H₃₃, C₁₇H₃₁, C₁₇H₂₉, C₁₉H₃₇, C₁₉H₃₅, C₁₉H₃₃, C₁₉H₃₁, or C₁₉H₂₉ and n can be 2, 3, or 4. In other examples, the polyamidoamines can have the chemical formula (O), where R¹ can be a laurate group, a stearate group, an isostearate group, an oleate group, a linoleate group, isomers thereof, or any mixture thereof and n can be 2, 3, or 4.

### EXAMPLES

In order to provide a better understanding of the foregoing discussion, the following non-limiting examples are offered. Although the examples can be directed to specific embodiments, they are not to be viewed as limiting the invention in any specific respect.

The cationic collectors contained polyamidoamines with varying types of hydrocarbyl groups on the amido groups and varying amounts and types of amine groups between the amido groups. The synergetic effects for selective phosphate flotation were due, at least in part, to these novel polyamidoamines to form the cationic collectors as highlighted by the results of Examples 1A-10B, summarized below in Table 1.

The synergetic effects for viscosity and homogeneity of the cationic collectors were due, at least in part, to the combination of the polyamidoamines, e.g., diamidoamines or triamidoamines, and acetic acid as highlighted by the results of Examples 11-41, summarized below in Tables 2 and 3. The cationic collectors contained varying amounts of acetic acid and water (when present) relative to a constant amount of polyamidoamines, and also contained varying polyamidoamine compositions within different cationic collectors. The synergetic effects for selective phosphate flotation were due, at least in part, to the combination of the diamidoamines and the acetic acid to form the cationic collectors as highlighted by the results of Examples 42A-47D, summarized below in Table 4.

Beneficiation Procedure:

The following phosphate beneficiation procedure was used for Examples 1A-10B and 42A-45D. About 500 g of phosphate rougher concentrate and about 214 g of water were added to a 2 L capacity stainless steel beaker equipped with a cruciform impeller. The concentrate and water were stirred for about 0.5 min and maintained at a pH value of about 7 (if needed, 1 N NaOH solution was added to adjust the pH value) to produce a mixture of about 70 wt % solids. For each of the Examples 1A-10B and 42A-45D, the listed polyamidoamine at the respective dosage was added to the mixture and stirred at about 400 rpm for about 5 min. The mixture was transferred to stainless steel flotation cell. About 1,300 g of water was added to the mixture that was stirred for about 0.5 min to produce a mixture of about 25 wt % solids. An air injection valve on the flotation cell was opened and frothing ensued as air was introduced into the mixture. After about 2 min, the froth was collected from the flotation cell. The froth concentrate and the tailings remaining in the flotation cell were separately filtered, dewatered, and weighed. The dried froth concentrate and the tailings were separately analyzed for phosphate (Bone Phosphate of Lime, BPL) content using inductively coupled plasma (ICP) and for acid insoluble content using an acid digestion.

### Examples 1A-1B

The TOFA-DETA polyamidoamine was made by the following: To a 40 mL scintillation vial equipped with a magnetic stir bar, about 20 g of tall oil fatty acid was added under an air atmosphere. The mixture was stirred and warmed to about 80° C., and about 3.68 g of DETA was added over about 4 min. The mixture exothermed to a temperature of about 105° C. to about 110° C., and then the mixture was heated for about 15 min to reach a temperature of about 165° C. As the reaction mixture neared 165° C., bubbling commenced, indicating reaction progress. The mixture was maintained at about 165° C. for about 3 hr, at which point, bubbling ceased. The mixture cooled to room temperature, e.g., about 25° C., and slowly formed a waxy substance.

### Examples 2A-2B

The lauric acid-TOFA-DETA polyamidoamine was made by the following: To a 40 mL scintillation vial equipped with a magnetic stir bar, about 10 g of tall oil fatty acid and about 6.95 g of lauric acid were added under an air atmosphere. The mixture was stirred and warmed to about 80° C., and about 3.7 g of DETA was added over about 4 min. The mixture exothermed to a temperature of about 105° C. to about 110° C., and then the mixture was heated for about 15 min to reach a temperature of about 165° C. As the reaction mixture neared 165° C., bubbling commenced, indicating reaction progress. The mixture was maintained at about 165° C. for about 3 hr, at which point, bubbling ceased. The mixture cooled to room temperature, e.g., about 25° C., and slowly formed a waxy substance.

### Examples 3A-3B

The naphthenic acid-TOFA-DETA polyamidoamine was made by the following: To a 40 mL scintillation vial equipped with a magnetic stir bar, about 10 g of tall oil fatty acid and about 8.125 g of naphthenic acid were added under an air atmosphere. The mixture was stirred and warmed to about 80° C., and about 3.7 g of DETA was added over about 4 min. The mixture exothermed to a temperature of about 105° C. to about 110° C., and then the mixture was heated for about 15 min to reach a temperature of about 165° C. As the reaction mixture neared 165° C., bubbling commenced, indicating reaction progress. The mixture was maintained at about 165° C. for about 3 hr, at which point, bubbling ceased. The mixture cooled to room temperature, e.g., about 25° C., and slowly formed a waxy substance.

### Examples 4A-4B

The isostearic acid-TOFA-DETA polyamidoamine was made by the following: To a 40 mL scintillation vial equipped with a magnetic stir bar, about 10 g of tall oil fatty acid and about 10.32 g of isostearic acid were added under an air atmosphere. The mixture was stirred and warmed to about 80° C., and about 3.7 g of DETA was added over about 4 min. The mixture exothermed to a temperature of about 105° C. to about 110° C., and then the mixture was heated for about 15 min to reach a temperature of about 165° C. As the reaction mixture neared 165° C., bubbling commenced, indicating reaction progress. The mixture was maintained at about 165° C. for about 3 hr, at which point, bubbling ceased. The mixture cooled to room temperature, e.g., about 25° C., and slowly formed a waxy substance.

### Examples 5A-5B

The LNI-TOFA-DETA polyamidoamine was made by the following: To a 40 mL scintillation vial equipped with a magnetic stir bar, about 12.5 g of tall oil fatty acid, about 1.74 g of lauric acid, about 2.22 g of naphthenic acid, and about 2.57 g of isostearic acid were added under an air atmosphere. The mixture was stirred and warmed to about 80° C., and about 3.68 g of DETA was added over about 4 min. The mixture exothermed to a temperature of about 105° C. to about 110° C., and then the mixture was heated for about 15 min to reach a temperature of about 165° C. As the reaction mixture neared 165° C., bubbling commenced, indicating reaction progress. The mixture was maintained at about 165° C. for about 3 hr, at which point, bubbling ceased. The mixture cooled to room temperature, e.g., about 25° C., and slowly formed a waxy substance.

### Examples 6A-6B

The TOFA-TEPA polyamidoamine was made by the following: To a 2 L reactor equipped with a mechanical stirrer, thermocouple, and Barrett trap/condenser, about 600 g of tall oil fatty acid was added under an air atmosphere. The mixture was stirred and warmed to about 80° C., and about 202 g of TEPA was added over about 4 min. The mixture exothermed to a temperature of about 105° C. to about 110° C., and then the mixture was heated for about 15 min to reach a temperature of about 165° C. The mixture was maintained at about 165° C. for about 3 hr, at which point, the Barrett trap had collected about 23 mL of water. The mixture cooled to room temperature, e.g., about 25° C., and slowly formed a waxy yellow substance.

### Examples 7A-7B

The lauric acid-TOFA-TEPA polyamidoamine was made by the following: To a 40 mL scintillation vial equipped with a magnetic stir bar, about 10 g of tall oil fatty acid and about 6.95 g of lauric acid were added under an air atmosphere. The mixture was stirred and warmed to about 80° C., and about 7.12 g of TEPA was added over about 4 min. The mixture exothermed to a temperature of about 105° C. to about 110° C., and then the mixture was heated for about 15 min to reach a temperature of about 165° C. As the reaction mixture neared 165° C., bubbling commenced, indicating reaction progress. The mixture was maintained at about 165° C. for about 3 hr, at which point, bubbling ceased. The mixture cooled to room temperature, e.g., about 25° C., and slowly formed a waxy substance.

### Examples 8A-8B

The naphthenic acid-TOFA-TEPA polyamidoamine was made by the following: To a 40 mL scintillation vial equipped with a magnetic stir bar, about 10 g of tall oil fatty acid and about 8.125 g of naphthenic acid were added under an air atmosphere. The mixture was stirred and warmed to about 80° C., and about 7.12 g of TEPA was added over about 4 min. The mixture exothermed to a temperature of about 105° C. to about 110° C., and then the mixture was heated for about 15 min to reach a temperature of about 165° C. As the reaction mixture neared 165° C., bubbling commenced, indicating reaction progress. The mixture was maintained at about 165° C. for about 3 hr, at which point, bubbling ceased. The mixture cooled to room temperature, e.g., about 25° C., and slowly formed a waxy substance.

### Examples 9A-9B

The isostearic acid-TOFA-TEPA polyamidoamine was made by the following: To a 40 mL scintillation vial equipped with a magnetic stir bar, about 10 g of tall oil fatty acid and about 10.32 g of isostearic acid were added under an air atmosphere. The mixture was stirred and warmed to about 80° C., and about 7.12 g of TEPA was added over about 4 min. The mixture exothermed to a temperature of about 105° C. to about 110° C., and then the mixture was heated for about 15 min to reach a temperature of about 165° C. As the reaction mixture neared 165° C., bubbling commenced, indicating reaction progress. The mixture was maintained at about 165° C. for about 3 hr, at which point, bubbling ceased. The mixture cooled to room temperature, e.g., about 25° C., and slowly formed a waxy substance.

### Examples 10A-10B

The LNI-TOFA-TEPA polyamidoamine was made by the following: To a 40 mL scintillation vial equipped with a magnetic stir bar, about 12.5 g of tall oil fatty acid, about 1.74 g of lauric acid, about 2.22 g of naphthenic acid, and about 2.57 g of isostearic acid were added under an air atmosphere. The mixture was stirred and warmed to about 80° C., and about 7.12 g of TEPA was added over about 4 min. The mixture exothermed to a temperature of about 105° C. to about 110° C., and then the mixture was heated for about 15 min to reach a temperature of about 165° C. As the reaction mixture neared 165° C., bubbling commenced, indicating reaction progress. The mixture was maintained at about 165° C. for about 3 hr, at which point, bubbling ceased. The mixture cooled to room temperature, e.g., about 25° C., and slowly formed a waxy substance.

In Examples 1A-10B, the flotation results listed in Table 1, demonstrates the effectiveness of collectors having diamidoamines used to remove impurities, such as acid insolubles, e.g., sand or silicate, from phosphate ore. The low acid insoluble values indicate removal of gangue from the crude phosphate mineral ore. The use of a mixed acid amidoamine system (a collector containing the amidoamines having the chemical formulas (E) and (G), where R¹ and R² were different hydrocarbyl groups, provided the low recovery of acid insoluble content in the phosphate concentrate. In both DETA and TEPA-based collectors, incorporation of naphthenic acid (or the naphthenate group) increased flotation of silicates, as shown in Table 1 for Examples 3A, 3B, 5A, 5B, 8A, 8B, 10A, and 10B. Surprisingly, these results indicate that the collectors can provide a technical and an economic benefit by removing impurities at lower dosages than does the traditional purely TOFA based system.

TABLE 1
Phosphate Beneficiation with Diamidoamines

| | | Dosage | P₂O₅ | A.I. | Separ. |
|---|---|---|---|---|---|
| | | (lb/ton) | Recov. | Recov. | Effic. |
| Ex. | Polyamidoamine | [kg/tonne] | (wt %) | (wt %) | (wt %) |
| 1A | TOFA-DETA | 1 [0.5] | 99.76 | 68.13 | 31.63 |
| 1B | TOFA-DETA | 2 [1] | 92.25 | 14.54 | 77.71 |
| 2A | lauric acid-TOFA- | 1 [0.5] | 99.88 | 94.18 | 5.7 |
| | DETA | | | | |
| 2B | lauric acid-TOFA- | 2 [1] | 92.75 | 11.66 | 81.09 |
| | DETA | | | | |
| 3A | naphthenic acid- | 1 [0.5] | 99.83 | 87.68 | 12.15 |
| | TOFA-DETA | | | | |
| 3B | naphthenic acid- | 2 [1] | 73.28 | 6.2 | 67.08 |
| | TOFA-DETA | | | | |
| 4A | isostearic acid- | 1 [0.5] | 99.83 | 81.74 | 8.09 |
| | TOFA-DETA | | | | |
| 4B | isostearic acid- | 2 [1] | 89.24 | 11.73 | 77.51 |
| | TOFA-DETA | | | | |
| 5A | LNI-TOFA-DETA | 1 [0.5] | 99.86 | 86.68 | 13.18 |
| 5B | LNI-TOFA-DETA | 2 [1] | 86.66 | 8.13 | 78.53 |
| 6A | TOFA-TEPA | 1 [0.5] | 99.16 | 28.16 | 71 |
| 6B | TOFA-TEPA | 2 [1] | 97.95 | 8.57 | 91.38 |
| 7A | lauric acid-TOFA- | 1 [0.5] | 98.54 | 53.28 | 45.26 |
| | TEPA | | | | |
| 7B | lauric acid-TOFA- | 2 [1] | 99.02 | 17.61 | 81.41 |
| | TEPA | | | | |
| 8A | naphthenic acid- | 1 [0.5] | 99.75 | 86.54 | 13.21 |
| | TOFA-TEPA | | | | |
| 8B | naphthenic acid- | 2 [1] | 94.99 | 7.23 | 87.76 |
| | TOFA-TEPA | | | | |
| 9A | isostearic acid- | 1 [0.5] | 99.8 | 78.69 | 21.11 |
| | TOFA-TEPA | | | | |
| 9B | isostearic acid- | 2 [1] | 98.64 | 10.12 | 88.52 |
| | TOFA-TEPA | | | | |
| 10A | LNI-TOFA-TEPA | 1 [0.5] | 99.45 | 30.69 | 68.76 |
| 10B | LNI-TOFA-TEPA | 2 [1] | 94.97 | 7.1 | 87.87 |

“LNI” is laurate, isostearate, and naphthenate groups

The TOFA-DETA polyamidoamines and the TOFA-TEPA polyamidoamines prepared in Examples 1A-1B and 6A-6B, were used in Examples 11-16 and 17-23, respectively.

### Example 11

The TOFA-DETA polyamidoamine acetate was prepared as follows: About 1 g of TOFA-DETA polyamidoamine was added to a 20 mL scintillation vial, then stirred and heated to about 80° C. About 0.093 g of glacial acetic acid was added over a period of about 2 min. The reaction mixture reached a temperature of about 100° C., then the heating source was removed and the mixture was cooled to about 25° C.

### Example 12

The TOFA-DETA polyamidoamine acetate was prepared as follows: About 1 g of TOFA-DETA polyamidoamine was added to a 20 mL scintillation vial, then stirred and heated to about 80° C. About 0.19 g of glacial acetic acid was added over a period of about 2 min. The reaction mixture reached a temperature of about 100° C., then the heating source was removed and the mixture was cooled to about 25° C.

### Example 13

The TOFA-DETA polyamidoamine acetate was prepared as follows: About 1 g of TOFA-DETA polyamidoamine was added to a 20 mL scintillation vial, then stirred and heated to about 80° C. About 0.28 g of glacial acetic acid was added over a period of about 2 min. The reaction mixture reached a temperature of about 100° C., then the heating source was removed and the mixture was cooled to about 25° C.

### Example 14

The TOFA-DETA polyamidoamine acetate was prepared as follows: About 1 g of TOFA-DETA polyamidoamine was added to a 20 mL scintillation vial, then stirred and heated to about 80° C. About 0.37 g of glacial acetic acid was added over a period of about 2 min. The reaction mixture reached a temperature of about 100° C., then the heating source was removed and the mixture was cooled to about 25° C.

### Example 15

The TOFA-DETA polyamidoamine acetate was prepared as follows: About 1 g of TOFA-DETA polyamidoamine was added to a 20 mL scintillation vial, then stirred and heated to about 80° C. About 0.37 g of glacial acetic acid and about 0.1 g of water were added over a period of about 2 min. The reaction mixture reached a temperature of about 100° C., then the heating source was removed and the mixture was cooled to about 25° C.

### Example 16

The TOFA-DETA polyamidoamine acetate was prepared as follows: About 1 g of TOFA-DETA polyamidoamine was added to a 20 mL scintillation vial, then stirred and heated to about 80° C. About 0.37 g of glacial acetic acid and about 0.2 g of water were added over a period of about 2 min. The reaction mixture reached a temperature of about 100° C., then the heating source was removed and the mixture was cooled to about 25° C.

### Example 17

The TOFA-TEPA polyamidoamine acetate was prepared as follows: About 1 g of TOFA-TEPA polyamidoamine was added to a 20 mL scintillation vial, then stirred and heated to about 80° C. About 0.23 g of glacial acetic acid was added over a period of about 2 min. The reaction mixture reached a temperature of about 100° C., then the heating source was removed and the mixture was cooled to about 25° C.

### Example 18

The TOFA-TEPA polyamidoamine acetate was prepared as follows: About 1 g of TOFA-TEPA polyamidoamine was added to a 20 mL scintillation vial, then stirred and heated to about 80° C. About 0.23 g of glacial acetic acid and about 0.25 g of water were added over a period of about 2 min. The reaction mixture reached a temperature of about 100° C., then the heating source was removed and the mixture was cooled to about 25° C.

### Example 19

The TOFA-TEPA polyamidoamine acetate was prepared as follows: About 1 g of TOFA-TEPA polyamidoamine was added to a 20 mL scintillation vial, then stirred and heated to about 80° C. About 0.23 g of glacial acetic acid and about 0.5 g of water were added over a period of about 2 min. The reaction mixture reached a temperature of about 100° C., then the heating source was removed and the mixture was cooled to about 25° C.

### Example 20

The TOFA-TEPA polyamidoamine acetate was prepared as follows: About 1 g of TOFA-TEPA polyamidoamine was added to a 20 mL scintillation vial, then stirred and heated to about 80° C. About 0.23 g of glacial acetic acid and about 0.75 g of water were added over a period of about 2 min. The reaction mixture reached a temperature of about 100° C., then the heating source was removed and the mixture was cooled to about 25° C.

### Example 21

The TOFA-TEPA polyamidoamine acetate was prepared as follows: About 1 g of TOFA-TEPA polyamidoamine was added to a 20 mL scintillation vial, then stirred and heated to about 80° C. About 0.44 g of glacial acetic acid was added over a period of about 2 min. The reaction mixture reached a temperature of about 100° C., then the heating source was removed and the mixture was cooled to about 25° C.

### Example 22

The TOFA-TEPA polyamidoamine acetate was prepared as follows: About 1 g of TOFA-TEPA polyamidoamine was added to a 20 mL scintillation vial, then stirred and heated to about 80° C. About 0.44 g of glacial acetic acid and about 0.25 g of water were added over a period of about 2 min. The reaction mixture reached a temperature of about 100° C., then the heating source was removed and the mixture was cooled to about 25° C.

### Example 23

The TOFA-TEPA polyamidoamine acetate was prepared as follows: About 1 g of TOFA-TEPA polyamidoamine was added to a 20 mL scintillation vial, then stirred and heated to about 80° C. About 0.9 g of glacial acetic acid was added over a period of about 2 min. The reaction mixture reached a temperature of about 100° C., then the heating source was removed and the mixture was cooled to about 25° C.

TABLE 2
Cationic Collectors with Polyamidoamines and Acetic Acid

| | | | | Viscosity | Viscosity | |
|---|---|---|---|---|---|---|
| | | HOAc | H₂O | at 80° C. | at 25° C. | |
| Ex. | Polyamidoamine | (g) | (g) | (cP) | (cP) | Homogeneity |
| 11 | TOFA-DETA | 0.093 | 0 | FF | HV | transparent |
| | | | | (30-125) | (1,500-3,500) | |
| 12 | TOFA-DETA | 0.19 | 0 | FF | HV | opaque |
| | | | | (30-125) | (900-2,500) | |
| 13 | TOFA-DETA | 0.28 | 0 | FF | HV | opaque, gel |
| | | | | (30-125) | (600-2,500) | |
| 14 | TOFA-DETA | 0.37 | 0 | FF | FF | transparent |
| | | | | (30-125) | (40-300) | |
| 15 | TOFA-DETA | 0.37 | 0.1 | FF | V | opaque, PS |
| | | | | (30-125) | (300-900) | |
| 16 | TOFA-DETA | 0.37 | 0.2 | FF | V | opaque, gel |
| | | | | (30-125) | (600-1,250) | |
| 17 | TOFA-TEPA | 0.23 | 0 | FF | HV | transparent |
| | | | | (30-125) | (900-2,500) | |
| 18 | TOFA-TEPA | 0.23 | 0.25 | FF | HV | opaque |
| | | | | (30-125) | (900-2,500) | |
| 19 | TOFA-TEPA | 0.23 | 0.5 | FF | HV | opaque, gel |
| | | | | (30-125) | (900-2,500) | |
| 20 | TOFA-TEPA | 0.23 | 0.75 | FF | HV | opaque, gel |
| | | | | (30-125) | (900-2,500) | |
| 21 | TOFA-TEPA | 0.44 | 0 | FF | V | transparent |
| | | | | (30-125) | (300-900) | |
| 22 | TOFA-TEPA | 0.44 | 0.25 | FF | HV | opaque |
| | | | | (30-125) | (900-2500) | |
| 23 | TOFA-TEPA | 0.9 | 0 | FF | FF | transparent |
| | | | | (30-125) | (40-300) | |

“FF” is free flowing;
“V” is viscous;
“HV” is highly viscous;
“NF” is no flow;
“PS” is phase separates

### Example 24

The LNI-TOFA-DETA polyamidoamine acetate was prepared as follows: About 1 g of LNI-TOFA-DETA polyamidoamine was added to a 20 mL scintillation vial, then stirred and heated to about 80° C. About 0.11 g of glacial acetic acid was added over a period of about 2 min. The reaction mixture reached a temperature of about 100° C., then the heating source was removed and the mixture was cooled to about 25° C.

### Example 25

The LNI-TOFA-DETA polyamidoamine acetate was prepared as follows: About 1 g of LNI-TOFA-DETA polyamidoamine was added to a 20 mL scintillation vial, then stirred and heated to about 80° C. About 0.21 g of glacial acetic acid was added over a period of about 2 min. The reaction mixture reached a temperature of about 100° C., then the heating source was removed and the mixture was cooled to about 25° C.

### Example 26

The LNI-TOFA-DETA polyamidoamine acetate was prepared as follows: About 1 g of LNI-TOFA-DETA polyamidoamine was added to a 20 mL scintillation vial, then stirred and heated to about 80° C. About 0.32 g of glacial acetic acid was added over a period of about 2 min. The reaction mixture reached a temperature of about 100° C., then the heating source was removed and the mixture was cooled to about 25° C.

### Example 27

The LNI-TOFA-DETA polyamidoamine acetate was prepared as follows: About 1 g of LNI-TOFA-DETA polyamidoamine was added to a 20 mL scintillation vial, then stirred and heated to about 80° C. About 0.42 g of glacial acetic acid was added over a period of about 2 min. The reaction mixture reached a temperature of about 100° C., then the heating source was removed and the mixture was cooled to about 25° C.

### Example 28

The LNI-TOFA-DETA polyamidoamine acetate was prepared as follows: About 1 g of LNI-TOFA-DETA polyamidoamine was added to a 20 mL scintillation vial, then stirred and heated to about 80° C. About 0.42 g of glacial acetic acid and about 0.1 g of water were added over a period of about 2 min. The reaction mixture reached a temperature of about 100° C., then the heating source was removed and the mixture was cooled to about 25° C.

### Example 29

The LNI-TOFA-DETA polyamidoamine acetate was prepared as follows: About 1 g of LNI-TOFA-DETA polyamidoamine was added to a 20 mL scintillation vial, then stirred and heated to about 80° C. About 0.42 g of glacial acetic acid and about 0.2 g of water were added over a period of about 2 min. The reaction mixture reached a temperature of about 100° C., then the heating source was removed and the mixture was cooled to about 25° C.

### Example 30

The LNI-TOFA-TEPA polyamidoamine acetate was prepared as follows: About 1 g of LNI-TOFA-TEPA polyamidoamine was added to a 20 mL scintillation vial, then stirred and heated to about 80° C. About 0.26 g of glacial acetic acid was added over a period of about 2 min. The reaction mixture reached a temperature of about 100° C., then the heating source was removed and the mixture was cooled to about 25° C.

### Example 31

The LNI-TOFA-TEPA polyamidoamine acetate was prepared as follows: About 1 g of LNI-TOFA-TEPA polyamidoamine was added to a 20 mL scintillation vial, then stirred and heated to about 80° C. About 0.37 g of glacial acetic acid was added over a period of about 2 min. The reaction mixture reached a temperature of about 100° C., then the heating source was removed and the mixture was cooled to about 25° C.

### Example 32

The LNI-TOFA-TEPA polyamidoamine acetate was prepared as follows: About 1 g of LNI-TOFA-TEPA polyamidoamine was added to a 20 mL scintillation vial, then stirred and heated to about 80° C. About 0.52 g of glacial acetic acid was added over a period of about 2 min. The reaction mixture reached a temperature of about 100° C., then the heating source was removed and the mixture was cooled to about 25° C.

### Example 33

The LNI-TOFA-TEPA polyamidoamine acetate was prepared as follows: About 1 g of LNI-TOFA-TEPA polyamidoamine was added to a 20 mL scintillation vial, then stirred and heated to about 80° C. About 0.52 g of glacial acetic acid and about 0.25 g of water were added over a period of about 2 min. The reaction mixture reached a temperature of about 100° C., then the heating source was removed and the mixture was cooled to about 25° C.

### Example 34

The lauric acid-TOFA-TEPA polyamidoamine acetate was prepared as follows: About 1 g of LNI-TOFA-TEPA polyamidoamine was added to a 20 mL scintillation vial, then stirred and heated to about 80° C. About 0.52 g of glacial acetic acid and about 1 g of water were added over a period of about 2 min. The reaction mixture reached a temperature of about 100° C., then the heating source was removed and the mixture was cooled to about 25° C.

### Example 35

The lauric acid-TOFA-TEPA polyamidoamine acetate was prepared as follows: About 1 g of lauric acid-TOFA-TEPA polyamidoamine was added to a 20 mL scintillation vial, then stirred and heated to about 80° C. About 0.53 g of glacial acetic acid was added over a period of about 2 min. The reaction mixture reached a temperature of about 100° C., then the heating source was removed and the mixture was cooled to about 25° C.

### Example 36

The lauric acid-TOFA-TEPA polyamidoamine acetate was prepared as follows: About 1 g of lauric acid-TOFA-TEPA polyamidoamine was added to a 20 mL scintillation vial, then stirred and heated to about 80° C. About 0.53 g of glacial acetic acid and about 0.1 g of water were added over a period of about 2 min. The reaction mixture reached a temperature of about 100° C., then the heating source was removed and the mixture was cooled to about 25° C.

### Example 37

The lauric acid-TOFA-TEPA polyamidoamine acetate was prepared as follows: About 1 g of lauric acid-TOFA-TEPA polyamidoamine was added to a 20 mL scintillation vial, then stirred and heated to about 80° C. About 0.53 g of glacial acetic acid and about 0.25 g of water were added over a period of about 2 min. The reaction mixture reached a temperature of about 100° C., then the heating source was removed and the mixture was cooled to about 25° C.

### Example 38

The lauric acid-TOFA-TEPA polyamidoamine acetate was prepared as follows: About 1 g of lauric acid-TOFA-TEPA polyamidoamine was added to a 20 mL scintillation vial, then stirred and heated to about 80° C. About 0.53 g of glacial acetic acid and about 1 g of water were added over a period of about 2 min. The reaction mixture reached a temperature of about 100° C., then the heating source was removed and the mixture was cooled to about 25° C.

### Example 39

The naphthenic acid-TOFA-TEPA polyamidoamine acetate was prepared as follows: About 1 g of naphthenic acid-TOFA-TEPA polyamidoamine was added to a 20 mL scintillation vial, then stirred and heated to about 80° C. About 0.53 g of glacial acetic acid and about 0.25 g of water were added over a period of about 2 min. The reaction mixture reached a temperature of about 100° C., then the heating source was removed and the mixture was cooled to about 25° C.

### Example 40

The isostearic acid-TOFA-TEPA polyamidoamine acetate was prepared as follows: About 1 g of isostearic acid-TOFA-TEPA polyamidoamine was added to a 20 mL scintillation vial, then stirred and heated to about 80° C. About 0.42 g of glacial acetic acid and about 0.25 g of water were added over a period of about 2 min. The reaction mixture reached a temperature of about 100° C., then the heating source was removed and the mixture was cooled to about 25° C.

### Example 41

The isostearic acid-TOFA-TEPA polyamidoamine acetate was prepared as follows: About 1 g of isostearic acid-TOFA-TEPA polyamidoamine was added to a 20 mL scintillation vial, then stirred and heated to about 80° C. About 0.53 g of glacial acetic acid and about 0.25 g of water were added over a period of about 2 min. The reaction mixture reached a temperature of about 100° C., then the heating source was removed and the mixture was cooled to about 25° C.

TABLE 3
Cationic Collectors with Polyamidoamines

| | | | | Viscosity | Viscosity | |
|---|---|---|---|---|---|---|
| | | HOAc | H₂O | at 80° C. | at 25° C. | |
| Ex. | Polyamidoamine | (g) | (g) | (cP) | (cP) | Homogeneity |
| 24 | LNI-TOFA- | 0.11 | 0 | FF | NF | transparent |
| | DETA | | | (30-125) | (1,500-3,500) | |
| 25 | LNI-TOFA- | 0.21 | 0 | FF | HV | opaque |
| | DETA | | | (30-125) | (900-2,500) | |
| 26 | LNI-TOFA- | 0.32 | 0 | FF | V | opaque, gel |
| | DETA | | | (30-125) | (300-900) | |
| 27 | LNI-TOFA- | 0.42 | 0 | FF | FF | transparent |
| | DETA | | | (30-125) | (40-300) | |
| 28 | LNI-TOFA- | 0.42 | 0.1 | FF | V | opaque, PS |
| | DETA | | | (30-125) | (300-900) | |
| 29 | LNI-TOFA- | 0.42 | 0.2 | FF | V | opaque, gel |
| | DETA | | | (30-125) | (100-600) | |
| 30 | LNI-TOFA- | 0.26 | 0 | FF | NF | transparent |
| | TEPA | | | (30-125) | (1,500-3,500) | |
| 31 | LNI-TOFA- | 0.37 | 0 | FF | NF | opaque |
| | TEPA | | | (30-125) | (1,500-3,500) | |
| 32 | LNI-TOFA- | 0.52 | 0 | FF | HV | opaque, gel |
| | TEPA | | | (30-125) | (600-2,500) | |
| 33 | LNI-TOFA- | 0.52 | 0.25 | FF | FF | transparent |
| | TEPA | | | (30-125) | (40-300) | |
| 34 | LNI-TOFA- | 0.52 | 1 | FF | V | opaque, PS |
| | TEPA | | | (30-125) | (100-600) | |
| 35 | lauric acid- | 0.53 | 0 | FF | HV | transparent |
| | TOFA-TEPA | | | (30-125) | (600-2,500) | |
| 36 | lauric acid- | 0.53 | 0.1 | FF | V | opaque |
| | TOFA-TEPA | | | (30-125) | (300-900) | |
| 37 | lauric acid- | 0.53 | 0.25 | FF | FF | transparent |
| | TOFA-TEPA | | | (30-125) | (40-300) | |
| 38 | lauric acid- | 0.53 | 1 | FF | V | opaque |
| | TOFA-TEPA | | | (30-125) | (100-600) | |
| 39 | naphthenic acid- | 0.53 | 0.25 | FF | FF | transparent |
| | TOFA-TEPA | | | (30-125) | (40-300) | |
| 40 | isostearic acid- | 0.42 | 0.25 | FF | V | gel-like |
| | TOFA-TEPA | | | (30-125) | (300-900) | |
| 41 | isostearic acid- | 0.53 | 0.25 | FF | FF | transparent |
| | TOFA-TEPA | | | (30-125) | (40-300) | |

“laur” is lauric acid;
“isos” is isostearic acid;
“naph” is naphthenic acid;
“LNI” is laurate, isostearate, and naphthenate groups;
“FF” is free flowing;
“V” is viscous;
“HV” is highly viscous;
“NF” is no flow;
“PS” is phase separates

### Examples 42A-42C

The TOFA-TEPA polyamidoamine acetate was prepared was prepared as follows: To a 2 L reactor, about 779 g of the TOFA-TEPA polyamidoamine was added, stirred, and heated to about 80° C. About 553 g of glacial acetic acid was added over a period of about 6 min. The reaction mixture reached a temperature of about 100° C., then the heating source was removed and the mixture was cooled to about 25° C.

### Examples 43A-43D

The TOFA-TEPA polyamidoamine acetate was prepared was prepared as follows: To a 2 L reactor, about 779 g of the TOFA-TEPA polyamidoamine was added, stirred, and heated to about 80° C. About 138 g of glacial acetic acid was added over a period of about 6 min. The reaction mixture reached a temperature of about 100° C., then the heating source was removed and the mixture was cooled to about 25° C.

### Examples 44A-44D

The lauric acid-TOFA-TEPA polyamidoamine acetate was prepared was prepared as follows: To a 2 L reactor, about 600 g of the lauric acid-TOFA-TEPA polyamidoamine was added, stirred, and heated to about 80° C. About 308 g of glacial acetic acid and 147 g of water were added over a period of about 6 min. The reaction mixture reached a temperature of about 100° C., then the heating source was removed and the mixture was cooled to about 25° C.

### Examples 45A-45D

The LNI-TOFA-TEPA polyamidoamine acetate was prepared was prepared as follows: To a 2 L reactor, about 600 g of the LNI-TOFA-TEPA polyamidoamine was added, stirred, and heated to about 80° C. About 299 g of glacial acetic acid and 143 g water were added over a period of about 6 min. The reaction mixture reached a temperature of about 100° C., then the heating source was removed and the mixture was cooled to about 25° C.

### Examples 46A-46D

The TOFA-DETA monoamidoamine acetate was prepared was prepared as follows: To a 2 L reactor, about 600 g of the TOFA-DETA monoamidoamine was added, stirred, and heated to about 80° C. About 210 g of glacial acetic acid, 600 g water, and 210 g Flomin 663 frother were added over a period of about 6 min. The reaction mixture reached a temperature of about 100° C., then the heating source was removed and the mixture was cooled to about 25° C.

### Examples 47A-47D

The cocoamine acetate was prepared was prepared as follows: To a 2 L reactor, about 600 g of the cocoamine was added, stirred, and heated to about 80° C. About 35.2 g of glacial acetic acid and 473 g water were added over a period of about 6 min. The reaction mixture reached a temperature of about 100° C., then the heating source was removed and the mixture was cooled to about 25° C.

TABLE 4
Phosphate Beneficiation

| | | | Dosage | P₂O₅ | A.I. | Separ. |
|---|---|---|---|---|---|---|
| | | HOAc | (lb/ton) | Recov. | Recov. | Effic. |
| Ex. | Polyamidoamine | (wt %)* | [kg/tonne] | (wt %) | (wt %) | (wt %) |
| 42A | TOFA-TEPA | 47 | 1 [0.5] | 99.75 | 49.14 | 50.61 |
| 42B | TOFA-TEPA | 47 | 1.5 [0.75] | 99.41 | 24.18 | 75.23 |
| 42C | TOFA-TEPA | 47 | 2 [1] | 96.64 | 8.13 | 88.52 |
| 43A | TOFA-TEPA | 18 | 1 [0.5] | 99.72 | 65.59 | 34.13 |
| 43B | TOFA-TEPA | 18 | 1.5 [0.75] | 98.52 | 11.65 | 86.88 |
| 43C | TOFA-TEPA | 18 | 2 [1] | 97.91 | 8.55 | 89.36 |
| 43D | TOFA-TEPA | 18 | 2.5 [1.25] | 97.37 | 8.05 | 89.32 |
| 44A | lauric acid-TOFA- | 30 | 1 [0.5] | 99.86 | 98.29 | 1.57 |
| | TEPA | | | | | |
| 44B | lauric acid-TOFA- | 30 | 1.5 [0.75] | 99.54 | 41.75 | 57.79 |
| | TEPA | | | | | |
| 44C | lauric acid-TOFA- | 30 | 2 [1] | 98.84 | 13.48 | 85.36 |
| | TEPA | | | | | |
| 44D | lauric acid-TOFA- | 30 | 2.5 [1.25] | 97.85 | 8.32 | 89.53 |
| | TEPA | | | | | |
| 45A | LNI-TOFA-TEPA | 30 | 1 [0.5] | 99.55 | 47.79 | 51.76 |
| 45B | LNI-TOFA-TEPA | 30 | 1.5 [0.75] | 99.46 | 43.17 | 56.29 |
| 45C | LNI-TOFA-TEPA | 30 | 2 [1] | 96.73 | 7.84 | 88.89 |
| 45D | LNI-TOFA-TEPA | 30 | 2.5 [1.25] | 94.91 | 9.18 | 85.73 |
| | Monoamidoamine | | | | | |
| 46A | TOFA-DETA | 13 | 1 [0.5] | 99.53 | 64.45 | 35.09 |
| | monoamidoamine | | | | | |
| 46B | TOFA-DETA | 13 | 1.5 [0.75] | 98.28 | 32.02 | 66.26 |
| | monoamidoamine | | | | | |
| 46C | TOFA-DETA | 13 | 2 [1] | 97.87 | 25.71 | 72.16 |
| | monoamidoamine | | | | | |
| 46D | TOFA-DETA | 13 | 2.5 [1.25] | 97.02 | 20.32 | 76.70 |
| | monoamidoamine | | | | | |
| 47A | cocoamine acetate | 6 | 1 [0.5] | 98.94 | 28.80 | 70.14 |
| 47B | cocoamine acetate | 6 | 1.5 [0.75] | 97.88 | 20.21 | 77.67 |
| 47C | cocoamine acetate | 6 | 2 [1] | 96.60 | 14.51 | 82.09 |
| 47D | cocoamine acetate | 6 | 2.5 [1.25] | 95.32 | 12.91 | 82.41 |

*HO Ac wt % values are based on the total weight of the polyamidoamine and the acetic acid.

Embodiments of the present disclosure further relate to any one or more of the following paragraphs:

1. A cationic collector, comprising a polyamidoamine, wherein the polyamidoamine comprises one or more amidoamines having the chemical formula (A), wherein R¹ and R² are each independently a saturated or unsaturated, substituted or unsubstituted, linear or branched, cyclic, heterocyclic, or aromatic hydrocarbyl group, and R¹ and R² are different hydrocarbyl groups, R³ and R⁴ are each independently a hydrogen or a saturated or unsaturated, substituted or unsubstituted, linear or branched, cyclic, heterocyclic, or aromatic hydrocarbyl group, each m is an integer of 1 to 5, and n is an integer of 2 to 8.

2. An aqueous mixture of a mineral ore, comprising a crude mineral ore, water, and a polyamidoamine, wherein the polyamidoamine comprises one or more amidoamines having the chemical formula (A), wherein R¹ and R² are each independently a saturated or unsaturated, substituted or unsubstituted, linear or branched, cyclic, heterocyclic, or aromatic hydrocarbyl group, and R¹ and R² are different hydrocarbyl groups, R³ and R⁴ are each independently a hydrogen or a saturated or unsaturated, substituted or unsubstituted, linear or branched, cyclic, heterocyclic, or aromatic hydrocarbyl group, each m is an integer of 1 to 5, and n is an integer of 2 to 8.

3. The aqueous mixture according to paragraph 2, wherein the crude mineral ore is a phosphorous ore, an iron ore, an aluminum ore, a potassium ore, a sodium ore, a calcium ore, potash, feldspar, bauxite, any mixture thereof.

4. An aqueous mixture of a phosphorous containing material, comprising a phosphorous ore, water, and a polyamidoamine, wherein the polyamidoamine comprises one or more amidoamines having the chemical formula (A), wherein R¹ and R² are each independently a saturated or unsaturated, substituted or unsubstituted, linear or branched, cyclic, heterocyclic, or aromatic hydrocarbyl group, and R¹ and R² are different hydrocarbyl groups, R³ and R⁴ are each independently a hydrogen or a saturated or unsaturated, substituted or unsubstituted, linear or branched, cyclic, heterocyclic, or aromatic hydrocarbyl group, each m is an integer of 1 to 5, and n is an integer of 2 to 8.

5. The aqueous mixture according to paragraph 4, wherein the polyamidoamine comprises one or more amidoamines having the chemical formula (D), wherein R¹ and R² are each independently C₁₁H₂₃, C₁₁H₂₁, C₁₅H₃₃, C₁₅H₃₁, C₁₅H₂₉, C₁₇H₃₅, C₁₇H₃₃, C₁₇H₃₁, or C₁₇H₂₉, and n is 2, 3, or 4, and wherein n is 2 and the polyamidoamine has a hydrophilic-lipophilic balance of about 7.5 to about 12, n is 3 and the polyamidoamine has a hydrophilic-lipophilic balance of about 16.5 to about 21, or n is 4 and the polyamidoamine has a hydrophilic-lipophilic balance of about 25.5 to about 30, based on the Davies' Method for hydrophilic-lipophilic balance.

6. A method for purifying a mineral, comprising combining crude mineral ore, water, and a polyamidoamine to produce an aqueous mixture, wherein the crude mineral ore is a silicate material, and wherein the polyamidoamine comprises one or more amidoamines having the chemical formula (A), wherein R¹ and R² are each independently a saturated or unsaturated, substituted or unsubstituted, linear or branched, cyclic, heterocyclic, or aromatic hydrocarbyl group, and R¹ and R² are different hydrocarbyl groups, R³ and R⁴ are each independently a hydrogen or a saturated or unsaturated, substituted or unsubstituted, linear or branched, cyclic, heterocyclic, or aromatic hydrocarbyl group, each m is an integer of 1 to 5, and n is an integer of 2 to 8, collecting a flocculated material comprising the silicate material and the polyamidoamine from the aqueous mixture, and collecting a purified mineral ore from the aqueous mixture.

7. The method according to paragraph 6, wherein n is 2 and the polyamidoamine has a hydrophilic-lipophilic balance of about 7.5 to about 12, n is 3 and the polyamidoamine has a hydrophilic-lipophilic balance of about 16.5 to about 21, or n is 4 and the polyamidoamine has a hydrophilic-lipophilic balance of about 25.5 to about 30, based on the Davies' Method for hydrophilic-lipophilic balance.

8. The method according to paragraph 6 or 7, wherein the polyamidoamine comprises a mixture of diamidoamines having a hydrophilic-lipophilic balance of about 7.5 to about 30, based on the Davies' Method for hydrophilic-lipophilic balance, wherein the mixture of diamidoamines comprises a first diamidoamine, a second diamidoamine, and a third diamidoamine, and wherein the first diamidoamine has a first chemical formula wherein n is 2, the second diamidoamine has a second chemical formula wherein n is 3, and the third diamidoamine has a third chemical formula wherein n is 4.

9. The method according to any one of paragraphs 6 to 8, wherein the purified mineral ore is a phosphorous ore, an iron ore, an aluminum ore, a potassium ore, a sodium ore, a calcium ore, potash, feldspar, bauxite, any mixture thereof.

10. The method according to any one of paragraphs 6 to 9, wherein the collected silicate material is about 90 wt % to about 99.99 wt % of the total silicate material contained in the crude mineral ore, and wherein the collected purified mineral ore is about 90 wt % to about 99.99 wt % of the total purified mineral ore contained in the crude mineral ore.

11. The cationic collector, the aqueous mixture, or the method according to any one of paragraphs 1 to 10, wherein R¹ is a C6 to C12 chain having 0 to 3 unsaturated bonds, R² is a C13 to C24 chain having 0 to 3 unsaturated bonds, R³ and R⁴ are hydrogen, each m is 2, 3, or 4, and n is 2, 3, 4, or 5.

12. The cationic collector, the aqueous mixture, or the method according to any one of paragraphs 1 to 11, wherein R¹ is a C10 to C24 chain, R² is a C1 to C5 chain, each m is 2, 3, or 4, and n is 2, 3, or 4.

13. The cationic collector, the aqueous mixture, or the method according to any one of paragraphs 1 to 12, wherein the polyamidoamine comprises one or more amidoamines having the chemical formula (D), wherein R¹ and R² are each independently C₉H₁₅, C₉H₁₃, C₁₁H₂₃, C₁₁H₂₁, C₁₅H₃₃, C₁₅H₃₁, C₁₅H₂₉, C₁₇H₃₅, C₁₇H₃₃, C₁₇H₃₁, or C₁₇H₂₉, and n is 2, 3, or 4.

14. The cationic collector, the aqueous mixture, or the method according to any one of paragraphs 1 to 13, wherein the polyamidoamine comprises one or more amidoamines having the chemical formula (O), wherein R¹ is a C6 to C24 chain, and n is 2, 3, or 4.

15. The cationic collector according to paragraph 14, wherein R¹ is a C10 to C24 chain having 0 to 2 unsaturated bonds, and n is 2, 3, or 4.

16. The cationic collector, the aqueous mixture, or the method according to any one of paragraphs 1 to 13, wherein the polyamidoamine comprises one or more amidoamines having the chemical formula (D), wherein R¹ is a C10 to C24 chain, R² is a C6 to C18 chain, and n is 2, 3, or 4.

17. The cationic collector according to paragraph 16, wherein R¹ is a C12 to C24 chain having 0 to 2 unsaturated bonds, R² is a C6 to C11 chain having 0 to 2 unsaturated bonds, and n is 2, 3, or 4.

18. The cationic collector, the aqueous mixture, or the method according to any one of paragraphs 1 to 17, wherein n is 2 and the polyamidoamine has a hydrophilic-lipophilic balance of about 7.5 to about 12, n is 3 and the polyamidoamine has a hydrophilic-lipophilic balance of about 16.5 to about 21, or n is 4 and the polyamidoamine has a hydrophilic-lipophilic balance of about 25.5 to about 30, based on the Davies' Method for hydrophilic-lipophilic balance.

19. The cationic collector, the aqueous mixture, or the method according to any one of paragraphs 1 to 18, wherein the polyamidoamine comprises a mixture of diamidoamines having a hydrophilic-lipophilic balance of about 7.5 to about 30, based on the Davies' Method for hydrophilic-lipophilic balance, wherein the mixture of diamidoamines comprises a first diamidoamine, a second diamidoamine, and a third diamidoamine, and wherein the first diamidoamine has a first chemical formula wherein n is 2, the second diamidoamine has a second chemical formula wherein n is 3, and the third diamidoamine has a third chemical formula wherein n is 4.

20. The cationic collector, the aqueous mixture, or the method according to any one of paragraphs 1 to 19, wherein the polyamidoamine comprises a polyethylene diamidoamine, a polyethylene triamidoamine, a polyethylene polyamidoamine with four or more amido groups, or any mixture thereof.

21. The cationic collector, the aqueous mixture, or the method according to any one of paragraphs 1 to 20, wherein the polyamidoamine comprises a mixture of polyethylene diamidoamines and polyethylene triamidoamines, and the mixture has about 0.5 mol % to about 20 mol % of the polyethylene triamidoamines, based on the combined moles of the polyethylene diamidoamines and the polyethylene triamidoamines.

22. The cationic collector, the aqueous mixture, or the method according to any one of paragraphs 1 to 21, wherein the cationic collector comprises an organic acid.

23. The cationic collector according to paragraph 22, wherein the cationic collector comprises about 10 wt % to about 60 wt % of the organic acid, based on a combined weight of the polyamidoamine and the organic acid, and wherein the organic acid comprises glycolic acid, lactic acid, pyruvic acid, formic acid, acetic acid, propionic acid, butyric acid, valeric acid, oxalic acid, isomers thereof, hydrates thereof, salts thereof, adducts thereof, or any mixture thereof.

24. The cationic collector according to paragraph 22, wherein the polyamidoamine is in an amount of about 40 wt % to about 90 wt %, based on a combined weight of the polyamidoamine and the organic acid, and wherein the organic acid is glacial acetic acid.

25. The cationic collector, the aqueous mixture, or the method according to any one of paragraphs 1 to 24, wherein the polyamidoamine comprises a product formed by reacting a polyamine and a fatty acid, wherein the polyamine comprises diethylenetriamine, triethylenetetramine, tetraethylenepentamine, pentaethylenehexamine, or any mixture thereof, and wherein the fatty acid comprises tall oil fatty acids, coconut oil fatty acids, lauric acid, stearic acid, isostearic acid, naphthenic acid, oleic acid, linoleic acid, linolenic acid, palmitic acid, isomers thereof, or any mixture thereof.

26. A cationic collector, comprising a polyamidoamine having the chemical formula (A), wherein: R¹ and R² are independently a saturated or unsaturated, substituted or unsubstituted, linear or branched, cyclic, heterocyclic, or aromatic hydrocarbyl group, R¹ and R² are different hydrocarbyl groups, each R³ is independently hydrogen or a saturated or unsaturated, substituted or unsubstituted, linear or branched, cyclic, heterocyclic, or aromatic hydrocarbyl group, R⁴ is hydrogen or a saturated or unsaturated, substituted or unsubstituted, linear or branched, cyclic, heterocyclic, or aromatic hydrocarbyl group each m is independently an integer of 1 to 5, and n is an integer of 2 to 8.

27. An aqueous mixture of a phosphorous containing material, comprising: an ore; water; and a polyamidoamine having the chemical formula (A), wherein: R¹ and R² are independently a saturated or unsaturated, substituted or unsubstituted, linear or branched, cyclic, heterocyclic, or aromatic hydrocarbyl group, R¹ and R² are different hydrocarbyl groups, each R³ is independently hydrogen or a saturated or unsaturated, substituted or unsubstituted, linear or branched, cyclic, heterocyclic, or aromatic hydrocarbyl group, R⁴ is hydrogen or a saturated or unsaturated, substituted or unsubstituted, linear or branched, cyclic, heterocyclic, or aromatic hydrocarbyl group, each m is independently an integer of 1 to 5, and n is an integer of 2 to 8.

28. A method for purifying a mineral, comprising: combining an ore, water, and a polyamidoamine to produce an aqueous mixture, wherein the ore is a silicate material, and wherein the polyamidoamine has the chemical formula (A), wherein: R¹ and R² are independently a saturated or unsaturated, substituted or unsubstituted, linear or branched, cyclic, heterocyclic, or aromatic hydrocarbyl group, R¹ and R² are different hydrocarbyl groups, each R³ is independently hydrogen or a saturated or unsaturated, substituted or unsubstituted, linear or branched, cyclic, heterocyclic, or aromatic hydrocarbyl group, R⁴ is hydrogen or a saturated or unsaturated, substituted or unsubstituted, linear or branched, cyclic, heterocyclic, or aromatic hydrocarbyl group, each m is independently an integer of 1 to 5, and n is an integer of 2 to 8; collecting a flocculated material comprising the silicate material and the polyamidoamine from the aqueous mixture; and collecting a purified ore from the aqueous mixture.

29. The cationic collector, the aqueous mixture, or the method according to any one of paragraphs 26 to 28, wherein: R¹ is a C6 to C12 chain having 0 to 3 unsaturated bonds, R² is a C13 to C24 chain having 0 to 3 unsaturated bonds, R⁴ and each R³ is hydrogen, each m is independently 2, 3, or 4, and n is 2, 3, 4, or 5.

30. The cationic collector, the aqueous mixture, or the method according to any one of paragraphs 26 to 28, wherein: R¹ is C₉H₁₅, C₉H₁₃, C₁₁H₂₃, C₁₁H₂₁, C₁₅H₃₃, C₁₅H₃₁, C₁₅H₂₉, C₁₇H₃₅, C₁₇H₃₃, C₁₇H₃₁, or C₁₇H₂₉, R² is C₉H₁₅, C₉H₁₃, C₁₁H₂₃, C₁₁H₂₁, C₁₅H₃₃, C₁₅H₃₁, C₁₅H₂₉, C₁₇H₃₅, C₁₇H₃₃, C₁₇H₃₁, or C₁₇H₂₉, R⁴ and each R³ is hydrogen, each m is 2, and n is 2, 3, or 4.

31. The cationic collector, the aqueous mixture, or the method according to any one of paragraphs 26 to 28, wherein the polyamidoamine has the chemical formula:

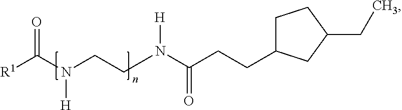

wherein: R¹ is a C6 to C24 chain, and n is 2, 3, 4, or 5.

32. The cationic collector, the aqueous mixture, or the method according to paragraph 31, wherein R¹ is a C10 to C24 chain having 0 to 2 unsaturated bonds, and n is 2, 3, or 4.

33. The cationic collector, the aqueous mixture, or the method according to any one of paragraphs 26 to 28, wherein: R¹ is a C10 to C24 chain, R² is a C6 to C18 chain, R⁴ and each R³ is hydrogen, each m is 2, and n is 2, 3, or 4.

34. The cationic collector, the aqueous mixture, or the method according to paragraph 33, wherein R¹ is a C12 to C24 chain having 0 to 2 unsaturated bonds and R² is a C6 to C11 chain having 0 to 2 unsaturated bonds.

35. A composition, comprising a polyamidoamine having the chemical formula:

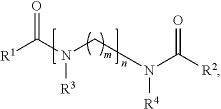

wherein: R¹ and R² are different and selected from a saturated or unsaturated, substituted or unsubstituted, linear or branched, cyclic, heterocyclic, or aromatic hydrocarbyl group, R³ and R⁴ are independently hydrogen or a saturated or unsaturated, substituted or unsubstituted, linear or branched, cyclic, heterocyclic, or aromatic hydrocarbyl group, each m is an integer of 1 to 5, and n is an integer of 2 to 8.

36. An aqueous mixture, comprising: an ore; water; and a polyamidoamine having the chemical formula:

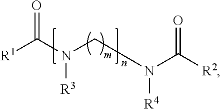

wherein: R¹ and R² are different and selected from a saturated or unsaturated, substituted or unsubstituted, linear or branched, cyclic, heterocyclic, or aromatic hydrocarbyl group, R³ and R⁴ are independently hydrogen or a saturated or unsaturated, substituted or unsubstituted, linear or branched, cyclic, heterocyclic, or aromatic hydrocarbyl group, each m is an integer of 1 to 5, and n is an integer of 2 to 8.

37. A method for purifying an ore, comprising: combining an ore, water, and a polyamidoamine to produce an aqueous mixture, wherein the ore comprises an impurity, and wherein the polyamidoamine has the chemical formula:

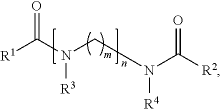

wherein: R¹ and R² are different and selected from a saturated or unsaturated, substituted or unsubstituted, linear or branched, cyclic, heterocyclic, or aromatic hydrocarbyl group, R³ and R⁴ are independently hydrogen or a saturated or unsaturated, substituted or unsubstituted, linear or branched, cyclic, heterocyclic, or aromatic hydrocarbyl group, each m is an integer of 1 to 5, and n is an integer of 2 to 8; collecting a flocculated material comprising the impurity and the polyamidoamine from the aqueous mixture; and collecting a purified ore having a reduced concentration of the impurity relative to the ore from the aqueous mixture.

38. The composition, aqueous mixture, or method according to any one of paragraphs 35 to 38, wherein: R¹ is a C6 to C12 chain having 0 to 3 unsaturated bonds, R² is a C13 to C24 chain having 0 to 3 unsaturated bonds, R³ and R⁴ are hydrogen, each m is 2, 3, or 4, and n is 2, 3, 4, or 5.

39. The composition, aqueous mixture, or method according to any one of paragraphs 35 to 38, wherein: R¹ is C₉H₁₅, C₉H₁₃, C₁₁H₂₃, C₁₁H₂₁, C₁₅H₃₃, C₁₅H₃₁, C₁₅H₂₉, C₁₇H₃₅, C₁₇H₃₃, C₁₇H₃₁, or C₁₇H₂₉, R² is C₉H₁₅, C₉H₁₃, C₁₁H₂₃, C₁₁H₂₁, C₁₅H₃₃, C₁₅H₃₁, C₁₅H₂₉, C₁₇H₃₅, C₁₇H₃₃, C₁₇H₃₁, or C₁₇H₂₉, R³ and R⁴ are hydrogen, each m is 2, and n is 2, 3, or 4.

40. The composition, aqueous mixture, or method according to any one of paragraphs 35 to 38, wherein the polyamidoamine has the chemical formula:

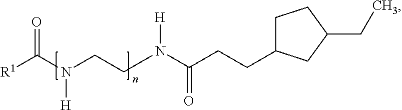

wherein: R¹ is a C6 to C24 chain, and n is 2, 3, 4, or 5.

41. The composition, aqueous mixture, or method according to any one of paragraphs 35 to 38 or 40, wherein R¹ is a C10 to C24 chain having 0 to 2 unsaturated bonds, and n is 2, 3, or 4.

42. The composition, aqueous mixture, or method according to any one of paragraphs 35 to 38, wherein: R¹ is a C10 to C24 chain, R² is a C6 to C18 chain, R³ and R⁴ are hydrogen, each m is 2, and n is 2, 3, or 4.

43. The composition, aqueous mixture, or method according to any one of paragraphs 35 to 38 or 42, wherein R¹ is a C12 to C24 chain having 0 to 2 unsaturated bonds and R² is a C6 to C11 chain having 0 to 2 unsaturated bonds.

44. The composition according to paragraph 35, wherein the composition is a cationic collector.

45. The cationic collector, composition, aqueous mixture, or method according to any one of paragraphs 26 to 44, wherein: n is 2 and the polyamidoamine has a hydrophilic-lipophilic balance of about 7.5 to about 12, as measured according to the Davies' Method for hydrophilic-lipophilic balance.

46. The cationic collector, composition, aqueous mixture, or method according to any one of paragraphs 26 to 44, wherein n is 3 and the polyamidoamine has a hydrophilic-lipophilic balance of about 16.5 to about 21, as measured according to the Davies' Method for hydrophilic-lipophilic balance.

47. The cationic collector, composition, aqueous mixture, or method according to any one of paragraphs 26 to 44, wherein n is 4 and the polyamidoamine has a hydrophilic-lipophilic balance of about 25.5 to about 30, as measured according to the Davies' Method for hydrophilic-lipophilic balance.

48. The cationic collector, composition, aqueous mixture, or method according to any one of paragraphs 26 to 47, wherein the polyamidoamine comprises a polyethylene diamidoamine, a polyethylene triamidoamine, a polyethylene polyamidoamine with four or more amido groups, or any mixture thereof.

49. The cationic collector, composition, aqueous mixture, or method according to any one of paragraphs 26 to 48, wherein the polyamidoamine comprises a mixture of polyethylene diamidoamines and polyethylene triamidoamines.

50. The cationic collector, composition, aqueous mixture, or method according to any one of paragraphs 26 to 49, wherein the mixture comprises about 0.5 mol % to about 20 mol % of the polyethylene triamidoamines, based on the combined moles of the polyethylene diamidoamines and the polyethylene triamidoamines.

51. The cationic collector, composition, aqueous mixture, or method according to any one of paragraphs 26 to 50, wherein the cationic collector further comprises an organic acid.

52. The cationic collector, composition, aqueous mixture, or method according to paragraph 51, wherein the cationic collector comprises about 10 wt % to about 60 wt % of the organic acid, based on a combined weight of the polyamidoamine and the organic acid.

53. The cationic collector, composition, aqueous mixture, or method according to paragraph 51 or 52, wherein the organic acid comprises glycolic acid, lactic acid, pyruvic acid, formic acid, acetic acid, propionic acid, butyric acid, valeric acid, oxalic acid, isomers thereof, hydrates thereof, salts thereof, adducts thereof, or any mixture thereof.

54. The cationic collector, composition, aqueous mixture, or method according to any one of paragraphs 51 to 53, wherein the mixture comprises about 40 wt % to about 90 wt % of the polyamidoamine, based on a combined weight of the polyamidoamine and the organic acid, and wherein the organic acid comprises glacial acetic acid.

55. The cationic collector, composition, aqueous mixture, or method according to any one of paragraphs 26 to 54, wherein the polyamidoamine comprises a product formed by reacting a polyamine and a fatty acid.

56. The cationic collector, composition, aqueous mixture, or method according to paragraph 55, wherein the polyamine comprises diethylenetriamine, triethylenetetramine, tetraethylenepentamine, pentaethylenehexamine, or any mixture thereof.

57. The cationic collector, composition, aqueous mixture, or method according to paragraph 55 or 56, wherein the fatty acid comprises tall oil fatty acids, coconut oil fatty acids, lauric acid, stearic acid, isostearic acid, naphthenic acid, oleic acid, linoleic acid, linolenic acid, palmitic acid, isomers thereof, or any mixture thereof.

58. The cationic collector, composition, aqueous mixture, or method according to any one of paragraphs 26 to 28 or 35 to 37, wherein R¹ and R² are independently C₁₁H₂₃, C₁₁H₂₁, C₁₅H₃₃, C₁₅H₃₁, C₁₅H₂₉, C₁₇H₃₅, C₁₇H₃₃, C₁₇H₃₁, or C₁₇H₂₉, R³ and R⁴ is hydrogen, and m is 2.

59. The cationic collector, composition, aqueous mixture, or method according to paragraph 58, wherein n is 2 and the polyamidoamine has a hydrophilic-lipophilic balance of about 7.5 to about 12, as measured according to the Davies' Method for hydrophilic-lipophilic balance.

60. The cationic collector, composition, aqueous mixture, or method according to paragraph 58, wherein n is 3 and the polyamidoamine has a hydrophilic-lipophilic balance of about 16.5 to about 21, as measured according to the Davies' Method for hydrophilic-lipophilic balance.

61. The cationic collector, composition, aqueous mixture, or method according to paragraph 58, wherein n is 4 and the polyamidoamine has a hydrophilic-lipophilic balance of about 25.5 to about 30, as measured according to the Davies' Method for hydrophilic-lipophilic balance.

62. The cationic collector, composition, aqueous mixture, or method according to any one of paragraphs 26 to 61, wherein the aqueous mixture further comprises acetic acid.

63. The aqueous mixture or the method according to any one of paragraphs 27 to 34 or 36 to 62, wherein the ore is a phosphorous ore, an iron ore, an aluminum ore, a potassium ore, a sodium ore, a calcium ore, potash, feldspar, bauxite, any mixture thereof.

64. The cationic collector, composition, aqueous mixture, or method according to any one of paragraphs 26, 29 to 34, or 45 to 63, wherein the cationic collector has a viscosity of about 10 cP to about 800 cP at a temperature of 25° C.

65. The cationic collector, the aqueous mixture, or the method according to any one of paragraphs 26, 29 to 34, or 45 to 63, wherein the cationic collector has a viscosity of about 10 cP to about 160 cP at a temperature of 25° C.

66. The cationic collector, the aqueous mixture, or the method according to any one of paragraphs 26, 29 to 34, or 45 to 63, wherein the cationic collector has a viscosity of about 10 cP to about 140 cP at a temperature of 25° C.

67. The cationic collector, the aqueous mixture, or the method according to any one of paragraphs 26, 29 to 34, or 45 to 63, wherein the cationic collector has a viscosity of about 10 cP to about 300 cP at a temperature of 80° C.

68. The cationic collector, the aqueous mixture, or the method according to any one of paragraphs 26, 29 to 34, or 45 to 63, wherein the cationic collector has a viscosity of about 10 cP to about 100 cP at a temperature of 80° C.

69. The cationic collector, the aqueous mixture, or the method according to any one of paragraphs 26, 29 to 34, or 45 to 63, wherein the cationic collector further comprises an organic acid and water, and wherein the cationic collector has a viscosity of about 10 cP to about 800 cP at a temperature of 25° C. when the cationic collector includes about 2 wt % to about 50 wt % of the organic acid, about 2 wt % to about 50 wt % of water, and about 30 wt % to about 95 wt % of the polyamidoamine, based on the combined weight of the polyamidoamine, the organic acid, and the water.

70. The cationic collector, the aqueous mixture, or the method according to any one of paragraphs 26, 29 to 34, or 45 to 63, wherein the cationic collector further comprises an organic acid and water, and wherein the cationic collector has a viscosity of about 10 cP to about 800 cP at a temperature of 25° C. when the cationic collector includes about 20 wt % to about 60 wt % of the organic acid, about 20 wt % to about 60 wt % of water, and about 30 wt % to about 80 wt % of the polyamidoamine, based on the combined weight of the polyamidoamine, the organic acid, and the water.

71. The composition according to any one of paragraphs 35 or 37 to 63, wherein the composition further comprises an organic acid and water, and wherein the composition has a viscosity of about 10 cP to about 800 cP at a temperature of 25° C. when the composition includes about 2 wt % to about 50 wt % of the organic acid, about 2 wt % to about 50 wt % of water, and about 30 wt % to about 95 wt % of the polyamidoamine, based on the combined weight of the polyamidoamine, the organic acid, and the water.

72. The composition according to any one of paragraphs 35 or 37 to 63, wherein the composition further comprises an organic acid and water, and wherein the composition has a viscosity of about 10 cP to about 800 cP at a temperature of 25° C. when the composition includes about 20 wt % to about 60 wt % of the organic acid, about 20 wt % to about 60 wt % of water, and about 30 wt % to about 80 wt % of the polyamidoamine, based on the combined weight of the polyamidoamine, the organic acid, and the water.

73. The aqueous mixture or method according to any one of paragraphs 36 to 70, wherein the ore is a phosphorous ore.

74. The aqueous mixture or method according to any one of paragraphs 36 to 70, wherein the impurity comprises a silicate material.

75. The aqueous mixture or method according to any one of paragraphs 36 to 70, 73, or 74, wherein the aqueous mixture comprises about 0.0001 wt % to about 2 wt % of the polyamidoamine, based on the weight of the ore.

76. The aqueous mixture or method according to any one of paragraphs 36 to 70, 73, or 75, wherein the aqueous mixture comprises about 0.0001 wt % to about 2 wt % of the organic acid, based on the weight of the ore.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments, other and further embodiments of the invention can be devised without departing from the basic scope thereof, and the scope thereof is determined by the claims that follow.

What is claimed is:

1. A composition, comprising a polyamidoamine having the chemical formula: 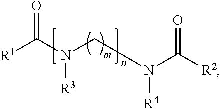
wherein:
R¹ and R² are different and selected from a saturated or unsaturated, substituted or unsubstituted, linear or branched, cyclic, heterocyclic, or aromatic hydrocarbyl group,
R³ and R⁴ are independently hydrogen or a saturated or unsaturated, substituted or unsubstituted, linear or branched, cyclic, heterocyclic, or aromatic hydrocarbyl group,
each m is an integer of 1 to 5, and
n is an integer of 2 to 8.

2. The composition of claim 1, wherein:
R¹ is a C6 to C12 chain having 0 to 3 unsaturated bonds,
R² is a C13 to C24 chain having 0 to 3 unsaturated bonds,
R³ and R⁴ are hydrogen,
each m is an integer of 2, 3, or 4, and
n is an integer of 2, 3, 4, or 5.

3. The composition of claim 1, wherein:
R¹ and R² are selected from C₉H₁₅, C₉H₁₃, C₁₁H₂₃, C₁₁H₂₁, C₁₅H₃₃, C₁₅H₃₁, C₁₅H₂₉, C₁₇H₃₅, C₁₇H₃₃, C₁₇H₃₁, or C₁₇H₂₉,
R³ and R⁴ are hydrogen,
each m is 2, and
n is 2, 3, or 4.

4. The composition of claim 1, wherein the polyamidoamine has the chemical formula: 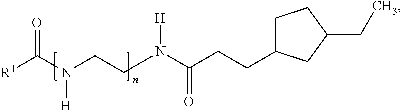
wherein:
R¹ is a C6 to C24 chain, and
n is 2, 3, 4, or 5.

5. The composition of claim 4, wherein R¹ is a C10 to C24 chain having 0 to 2 unsaturated bonds, and n is 2, 3, or 4.

6. The composition of claim 1, wherein:
R¹ is a C10 to C24 chain,
R² is a C6 to C18 chain,
R³ and R⁴ are hydrogen,
each m is 2, and
n is 2, 3, or 4.

7. The composition of claim 6, wherein R¹ is a C12 to C24 chain having 0 to 2 unsaturated bonds and R² is a C6 to C11 chain having 0 to 2 unsaturated bonds.

8. The composition of claim 1, wherein:
n is 2 and the polyamidoamine has a hydrophilic-lipophilic balance of about 7.5 to about 12, as measured according to the Davies' Method for hydrophilic-lipophilic balance;
n is 3 and the polyamidoamine has a hydrophilic-lipophilic balance of about 16.5 to about 21, as measured according to the Davies' Method for hydrophilic-lipophilic balance; or
n is 4 and the polyamidoamine has a hydrophilic-lipophilic balance of about 25.5 to about 30, as measured according to the Davies' Method for hydrophilic-lipophilic balance.

9. The composition of claim 1, wherein the polyamidoamine comprises a polyethylene diamidoamine, a polyethylene triamidoamine, a polyethylene polyamidoamine with four or more amido groups, or any mixture thereof.

10. The composition of claim 1, wherein the polyamidoamine comprises a mixture of polyethylene diamidoamines and polyethylene triamidoamines, and wherein the mixture comprises about 0.5 mol % to about 20 mol % of the polyethylene triamidoamines, based on the combined moles of the polyethylene diamidoamines and the polyethylene triamidoamines.

11. The composition of claim 1, wherein the composition further comprises an organic acid.

12. The composition of claim 11, wherein the composition comprises about 10 wt % to about 60 wt % of the organic acid, based on a combined weight of the polyamidoamine and the organic acid, and wherein the organic acid comprises glycolic acid, lactic acid, pyruvic acid, formic acid, acetic acid, propionic acid, butyric acid, valeric acid, oxalic acid, isomers thereof, hydrates thereof, salts thereof, adducts thereof, or any mixture thereof.

13. The composition of claim 11, wherein the composition comprises about 40 wt % to about 90 wt % of the polyamidoamine, based on a combined weight of the polyamidoamine and the organic acid, and wherein the organic acid comprises glacial acetic acid.

14. The composition of claim 1, wherein the polyamidoamine comprises a product formed by reacting a polyamine and a fatty acid, wherein the polyamine comprises diethylenetriamine, triethylenetetramine, tetraethylenepentamine, pentaethylenehexamine, or any mixture thereof, and wherein the fatty acid comprises tall oil fatty acids, coconut oil fatty acids, lauric acid, stearic acid, isostearic acid, naphthenic acid, oleic acid, linoleic acid, linolenic acid, palmitic acid, isomers thereof, or any mixture thereof.

15. An aqueous mixture, comprising:
an ore;
water; and
a polyamidoamine having the chemical formula: 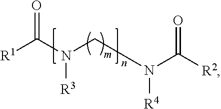
wherein:
R¹ and R² are different and selected from a saturated or unsaturated, substituted or unsubstituted, linear or branched, cyclic, heterocyclic, or aromatic hydrocarbyl group,
R³ and R⁴ are independently hydrogen or a saturated or unsaturated, substituted or unsubstituted, linear or branched, cyclic, heterocyclic, or aromatic hydrocarbyl group,
each m is an integer of 1 to 5, and
n is an integer of 2 to 8.

16. The aqueous mixture of claim 15, wherein:
R¹ and R² are independently C₁₁H₂₃, C₁₁H₂₁, C₁₅H₃₃, C₁₅H₃₁, C₁₅H₂₉, C₁₇H₃₅, C₁₇H₃₃, C₁₇H₃₁, or C₁₇H₂₉,
R³ and R⁴ are hydrogen, and
each m is 2, and wherein:
n is 2 and the polyamidoamine has a hydrophilic-lipophilic balance of about 7.5 to about 12, as measured according to the Davies' Method for hydrophilic-lipophilic balance;
n is 3 and the polyamidoamine has a hydrophilic-lipophilic balance of about 16.5 to about 21, as measured according to the Davies' Method for hydrophilic-lipophilic balance; or
n is 4 and the polyamidoamine has a hydrophilic-lipophilic balance of about 25.5 to about 30, as measured according to the Davies' Method for hydrophilic-lipophilic balance.

17. The aqueous mixture of claim 15, wherein the aqueous mixture further comprises acetic acid, wherein the ore is a phosphorous ore, an iron ore, an aluminum ore, a potassium ore, a sodium ore, a calcium ore, potash, feldspar, bauxite, any mixture thereof, and wherein the aqueous mixture comprises about 0.0001 wt % to about 2 wt % of the polyamidoamine and about 0.0001 wt % to about 2 wt % of the acetic acid, based on the weight of the ore.

18. A method for purifying an ore, comprising:
combining an ore, water, and a polyamidoamine to produce an aqueous mixture, wherein the ore comprises an impurity, and wherein the polyamidoamine has the chemical formula: 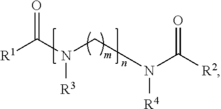
wherein:
R¹ and R² are different and selected from a saturated or unsaturated, substituted or unsubstituted, linear or branched, cyclic, heterocyclic, or aromatic hydrocarbyl group,
R³ and R⁴ are independently hydrogen or a saturated or unsaturated, substituted or unsubstituted, linear or branched, cyclic, heterocyclic, or aromatic hydrocarbyl group,
each m is an integer of 1 to 5, and
n is an integer of 2 to 8;
collecting a flocculated material comprising the impurity and the polyamidoamine from the aqueous mixture; and
collecting a purified ore having a reduced concentration of the impurity relative to the ore from the aqueous mixture.

19. The method of claim 18, wherein:
n is 2 and the polyamidoamine has a hydrophilic-lipophilic balance of about 7.5 to about 12, as measured according to the Davies' Method for hydrophilic-lipophilic balance;
n is 3 and the polyamidoamine has a hydrophilic-lipophilic balance of about 16.5 to about 21, as measured according to the Davies' Method for hydrophilic-lipophilic balance; or
n is 4 and the polyamidoamine has a hydrophilic-lipophilic balance of about 25.5 to about 30, as measured according to the Davies' Method for hydrophilic-lipophilic balance.

20. The method of claim 18, wherein the ore is a phosphorous ore, an iron ore, an aluminum ore, a potassium ore, a sodium ore, a calcium ore, potash, feldspar, bauxite, any mixture thereof, wherein the impurity comprises a silicate material, and wherein the aqueous mixture comprises about 0.0001 wt % to about 2 wt % of the polyamidoamine, based on the weight of the ore.
